SEQUENCE LISTING (81 sequences)

SEQ ID NO: 1 - protein, 208 aa, Acremonium alcalophilum
RIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 2 - protein, 213 aa, Acremonium alcalophilum
SPIRRRIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 3 - protein, 218 aa, Aspergillus fumigatus
LPSQPEARATTVQGFDISNHQKSVNFEAAKKDGAQFVMIKATEGTTYKDTVFNSHYTGATKAGLLRGGYHFARPDKSTGSTQAKFFLKNGGGWSDDNRTLPGMLDIEYNPYGATCYGLSHSQMVAWIHDFVNEYHHATSRWPMIYTTADWWNRCTGNAKGFGDKCPLVLAAYSSSPPKTIPGDWKTWTIWQNSDKYKHGGDSDKFNGPMTQLRKLASG

SEQ ID NO: 4 - protein, 208 aa, Trichoderma reesei
TVPGFDISHYQATVDFAKAYADGARFVIIKATEGTTYTDPSFSDHYTKATNAGFIRGGYHFAQPASSSGAAQANYFLKHGGGWSADGITLPGMLDLEYAPSGDSCYGLSASAMVSWINDFVNTYHAATTQYPLIYTSTSWWQLCTGNNGSFGSKSPLVIARYASSVGALPNGWSVYTIWQNSDASPWGGDNDIFNGNLAQLQKIARGS

SEQ ID NO: 5 - protein, 215 aa, Trametes cinnabarina
SPTPEKRANPKGIDVSAYQPNINWSTVKANGISFAYIKATEGTTYTNPDFSSQYTGATNAGLIRGGYHFAHPDSSSGATQAKYFLAHGGGWTSDGITLPGALDIEYNPSGAECYGLSASAMVSWIKDFSNTYHSSTGVYPVIYTTTDWWTTCTGNSAAFASTNPLWIARYASSIGTLPAGWSYTTFWQYADSGPNPGDQDEFNGSMAGLKQLALG

SEQ ID NO: 6 - protein, 207 aa, Sporormia fimetaria
AVPGFDISHYQSSVNYAGAYSSGARFVIIKATEGTTYIDPKFSDHYIGATNAGLIRGAYHFARPAASTGAAQANYFVSHGGGWSADGITLPGMLDMEYGSTSACHGLSQSAMVTWITSFVNQYNSLTGRYPMIYTTADWWQTCTGNSAAFNTKSPLVLARYSSSAGTVPGGWPYYTIWQFNDAYAYGGDSDTFNGDLAGLKRLAKGS

SEQ ID NO: 7 - protein, 201 aa, Poronia punctata
AVQGFDISHYQSSVNFGAAKSSGAQFVIIKATEGTSYTDPSFSSHYTGATNAGLIRGGYHFAHLDSSSGAAQAKYFLAHGGGWSGDGITLPGMLDLEGSCVLSASATVSWIKDFSNTYHSSTGVYPLIYTNPSWWSSCTGNSKAFIDTNPLVLARYASSAGTPPGGWPYYTIWQYNDAYKYGGDSDVFNGDLAGLKRLAKG

SEQ ID NO: 8 - protein, 201 aa, Poronia punctata
QVQGFDISSYQPSVDFAGAYADGARFVIIKATEGTGYIDPTFSDHYVGATNAGLLRGGYHYAHLDSTSGATQAQYFLANGGGWSGDGITLPGMLDLEGDCVLSAADAVAWIKDFSDTYHASTGVYPLLYTNPSWWASCTGDSSAFIDTNPLVLAHYADAAGTPPGGWPFYSFWQYNDAYPYGGDSEVWNGDMDGLLRLASG

SEQ ID NO: 9 - protein, 203 aa, Lecanicillium sp. WMM742
VDSSSEVSVAIYKKALGQGFTRAIFRGYQEACSQGGRVDPTFVPSYKNAVAAGYKDFDAYFFPCTGKTNKCKPYAAQLAELLDTIKGQKLAIRRIWLDIETDRVCNPFDYGAQGNLAEAKKLVAAFRDAKLDWGIYTSPTQWETIFGAKTVELAKDVPLWFAKFDNVETLELKTPFGGWTKADAKQYTDQSASNKFDLNVFSA

SEQ ID NO: 10 - protein, 208 aa, Lecanicillium sp. WMM742
TVQGFDVSGYQPTVNWGAAYSSGARFVMIKATEGTGYISSSFGSQYPGATNAGFIRGGYHFALPDRSSGSAQADYFLAHGGGWSGDGITLPGMLDIEYNPYGATCYGLSQGAMVNWISDFVEHYKARTTQYPIIYTTTDWWKTCTGNSPAFGQKCPLSLARYSSSVGEIPNGWPFQTFWQNSDKYAYGGDSQIFNGAYSQLQKIARGG

SEQ ID NO: 11 - protein, 207 aa, Onygena equina
AVPGIDVSGYQGNVNWANVANAGKKFAYVKATEHTNYINPYFAQQYNGAYNQGIIRGAYHYAHPNGASGASQANYFLAHGGGWSADGKTLPGAVDLEYGPNGSTCWGISQSAMIAWIRDFSNTYRAKTGRPPVIYTSTSWWKTCTGNYGGFGNDNPLWIARYSSTVGELPAGWPFHSIWQNNDNSGVGGDGDIWNGDLAGLQRFAKG

SEQ ID NO: 12 - protein, 208 aa, Purpureocillium lilacinum
AVKGFDISHYQPNVDFAKAYADGARFVMIKATEGTTYTDPSFSSHYTGATKAGFIRGGYHFARPASSSGAAQAKYFIAHGGGWSKDGITLPGMLDMEYQSSSSACGGLSQSAMVSWINDFVNTYHAATGVYPLIYTSTSWWTQCTGNSAAFGSKCPLVVARYASSVGTLPAGWGFYTFWQYSDAAPWGGDADTFNGDITALKKIANAG

SEQ ID NO: 13 - protein, 207 aa, Trichobolus zukalii
AVPGFDISHYQPSVNYAGAYNSGARFVIIKATEGTTYTDPVFSTHYTGATKAGLIRGGYHFARPASSSGSAQADFFFKNGGGWSADGITLPGMLDMEYGSTSSCHGLSQTAMVNWISDFVNRYKTLSGRYPMIYTGYYWWVECTGNSNKFATTCPLVLARYSSSVGEIPGGWGYQTIWQFNDKYAYGGDSDSFNGSLDRLKALAKGT

SEQ ID NO: 14 - protein, 207 aa, Penicillium citrinum
LIHAVDSSSEVSVDIYKKALSEGFSRAIFRGYQEACSQGGRVDPTFLPSYKNAQTAGYKDFDAYFFPCTGSGNKCKPYDVQIGELVDAIKKNNMAIRRIWVDFEKDKTCNPFNWDPKRNIDEAKRIIGAVRKTKFDFGVYTSATQWTSIFGSKDVVLANDVPLWFAKFDNVENLDLAQPFGGWTKADGKQYTDKSASKKFDLNVFSA

SEQ ID NO: 15 - protein, 207 aa, Cladorrhinum bulbillosum
AVQGFDISHYQSSVNFQAAYNSGARFVIIKATEGTTYIDPKFSSHYTGATNAGLIRGGYHFAHPDSSTGAAQADFFLAHGGGWSGDGITLPGMLDLESVSGKATCFGLSASSMVAWIKSFSDRYHTRTGRYPMLYTNPSWWTTCTGNSNAFVNTNPLVLARYASAPGTIPGGWPYQTIWQNSDSYTYGGDSDIFNGALSGLQKLASG

SEQ ID NO: 16 - protein, 208 aa, Umbelopsis westeae
KLKGLDVSGYQPNVAWSTVKANGASFAYIKATEGTNYKNPSFAQQYNGAYNAGLIRGSYHFAQPSSSTGAAQANYFLAHGGGWSPDGKTLPGALDMEYNPHGSTCYGLSKDAMVKWIKDFSNTYHSATGRYPVIYTTTSWWTTCTGNSAAFGATNPLWIARYSSTAGNLPNGWAFYSFWQNADSGIFPGDQDIWNGDAAALSRMAKGA

SEQ ID NO: 17 - protein, 208 aa, Zygomycetes sp. XZ2655
TLPGLDVSSYQGNVNWGTVASQGAKFAYVKATEGTTYTNPYFASQYDGSYNAGLIRGAYHFAHPDSSSGATQANYFLAHGGGWSADGKTLPGALDIEYNPNGAECYGLSQLAMISWIQDFSNTYHSHTGRYPVIYTTTDWWTTCTGNSAAFGTNNPLWIARYSSSVGTLPAGWGYESFWQKASSGTFPGDQDIWNGDAAGLSRFATGK

SEQ ID NO: 18 - protein, 206 aa, Chaetomium cupreum
TVQGFDISGYQPNVNFAAAYAAGARFVIIKATEGTSYISPSFSSQYTGATNAGFIRGGYHFAHPGASSGTTQADYFIAHGGGWTPDGITLPGMLDLESESSGTCWGLSASAMVAWIKDFSDHYHSRMGVYPLLYTNPSWWEECTGNSNAFVDTNPLVLAHYSSSVGTIPGGWPYETIWQNSDSYAYGGDSDVFNGDLAGLQRLARG

SEQ ID NO: 19 - protein, 207 aa, Cordyceps cardinalis
LIHAVDSSSEVSVDVYKKALAEGFTRAIFRGYQEACSQGGRVDPTFLPSYKNAQKAGYKDFDAYFFPCTGSGNKCKPYAKQIGELVDAIEGNQLAIRRIWIDIETDKVCNAFNWGAEGNIQEAKKLIAAVRGTKRDFGIYTSATQWENIFGSRTVELAKDVPLWFAKFDNVETLELKTPFGGWTKADAKQYTDKSASKKFDLNVFSA

SEQ ID NO: 20 - protein, 216 aa, Penicillium sp. ′qii′
STIQPRASGVQGFDISSYQGTVNFAGAYGAGARFVMIKATEGTTYIDSTFSSHYDGATSAGLIRGAYHFAHPDSSSGATQAEYFLAHGGGWTNDGITLPGMLDIEYNPSGSTCYGLSASAMVSWIKDFGETYNSKTGRYPMIYSTADWWSTCTGDSTSFSSDYPLVLAQYASSISTVPGGWPYQSFWQNADSYSYGGDSDLWNGSEDSLKTFAKGS

SEQ ID NO: 21 - protein, 218 aa, Aspergillus sp. nov XZ2609
LPTKLAARYSTVQGFDVSNYQPNVDFSAAKSAGAEFVIIKATEGTDYKDTYFNSHYTGATNAGLIRGGYHFARPDKSSGTAQAEYFLAHGGGWSKDGRTLPGMLDIEYNPYGATCYGLSHSAMVSWVNEFLNEYHSKTGVYPLLYTTADWWNQCTGNAHGFGNKSPLVLASYSSESPRTVPGDWQTWTIWQNADKYKYGGDSDIFNGDLTQLKKIVEG

SEQ ID NO: 22 - protein, 204 aa, Paecilomyces sp. XZ2658
TVAGFDISNYQPSVNFAKAYADGARFATEGTTYIDPSFSSHYTGATNAGLIRGGYHFAHPGSSTGAAQATYFLAHGGGWSKDGITLPGMIDLEYNPSGATCYGLSTSAMVSWISDFVETYHSKTGVYPLIYTSTSWWNQCTGSSTAFASKCPLVVARYASSVGTLPAGWGYQTIWQNSDSSPWGGDNDIFNGSLDQLKRIANAS

SEQ ID NO: 23 - protein, 203 aa, Paecilomyces sp. XZ2658
AVQGHDVSHWQGNINWGAVKAAGVKFTYIKATESTNYIDPSFNANYVGATNTGLIRGAYHFARPGDSSGAAQANYFVSHGGGWSADGRTLPGALDLEAGCSGLSQSAMTAWIRDFSNTYHARTGRFPVIYTTTSWWKTCTGNASGFQNDHPLWIARWGPSPGELPAGYGFHTFWQYADKGPLPGDQDNFNGDEAGLARLARGS

SEQ ID NO: 24 - protein, 208 aa, Pycnidiophora cf dispera
AVSGMDISHYQGTNYNFAGAYSSGARFVIIKATEGTTYTDPQFSANYIGATNAGFIRGAYHFARPAASTGAVQASYFVSHGGGWSSDGITLPGMLDMEYGSTSTCHGLSVSAMNTWIASFVNQYKSLTGAYPMIYTTADWWKTCTGDSTAWNTKCPLVLARYSSSVGTIPGGWPYHTIWQYSDSYAYGGDSDTFNGDLAGLKRLAKGS

SEQ ID NO: 25 - protein, 210 aa, Thermomucor indicae-seudaticae
YQTGLDVSALTSTSSFSCAKNLGYDHVIARCYMEAYGNNPGGKVDPNCYSNYKNAKAAGFTSVDIYMFPCTGRSTCKSPATQVQEIVDYVGAHKMIVGTLWLDVEVDSAANNWPSTSEARSTLRAFKTALDKSGWKWGVYSSKSQWTRITGSASWVLDPSVPLWYSHYDDTLSFSDYPSHAFGGWSKPTIKQYTGDASFCSASWDKNYYG

SEQ ID NO: 26 - protein, 207 aa, Isaria farinosa
LTHAVDSSSEVSVDIYKKALGQGFTRAIFRGYQEACSLGGRVDPTFVPSYKNAVAAGYKDFDAYFFPCTGTTNKCKPYATQLAELLDTISSQKLAIRRIWLDIETDQVCSPFDYGAQGNIAEAKKLVAAFRAAKHDWGIYTSPTQWETIFGSKTFVLANDVPLWFAKFDNVETLDLKTPFGGWTKADAKQYTDQSASKKFDLNVFSA

SEQ ID NO: 27 - protein, 207 aa, Lecanicillium sp. WMM742
SVQGFDISHYQSSVNFGAAYADGARFVIIKATEGTTYRDPKFSEHYGGATKAGFIRGGYHFAQPASSSGAAQANFFLAHGGGWSGDGITLPGMLDLEYGPNGNTCYGLGPASMRSWISDFVETYHAKTGRYPLIYTSTSWWKTCTGNTSLFADKCPLVVARYNSQVGELPAGWGFYTFWQFNDHYKHGGDSDVFNGAYSQLQKIATG

SEQ ID NO: 28 - protein, 208 aa, Zopfiella sp. t180-6
AVQGFDVSHWQSSVNFAAAYNSGARFVIIKATESNNYIDPKFNTYYPAATSAGLIRGGYHFAHPGETTGAVQADYFIAHGGGWSSDGITLPGMLDLENASGYPACWGLSQSAMVSWIKAFSDRYKARTGVYPMLYTNPSWWTSCTGNSNAFVNTNPLVLARYASSPGTIPGGWPYQTIWQNSDSYTYGGDSDIFNGDLAGLKRLAKGS

SEQ ID NO: 29 - protein, 217 aa, Malbranchea flava
GPKEFESRASGVQGFDISGWQSNVNFAGAYNSGARFVMIKASEGTTFKDRQFSNHYIGATKAGFIRGGYHFALPDVSSATAQVNHFLASGGGWSRDGITLPGMLDIESNPYGAQCYGLDAGRMVAWIREFVDAYKRATGRYPLIYTSPSWWQTCTGNSNAFIDKCPLVLARWASSPGTPPGGWPFHSFWQYADSYQFGGDAQVFNGDEAGLKRMALG

SEQ ID NO: 30 - protein, 208 aa, Hypholoma polytrichi
LVYGVDSSSLVPVATYQKALGEGFTKAVIRGYEEACGVGGEVDPNFVPSYKNARAAGYTDIDMYWFPCNGSTHSCKSYAAQLAAIAAAFSANAMKIGTIWIDIEKDAAICNNWDYGTAGNLAQAKALIAAAKASGFNFGIYSSPGEWSTIFGSTSVVVDNSAPLWFATYNNVQTLTLGTPFGGWSTAVGHQYTDVSASGLFDLNVFAH

SEQ ID NO: 31 - protein, 201 aa, Aspergillus deflectus
AVQGFDISHYQSSVDYAGAYSSGARFVMIKATEGTTYTDPAFSTHYTGATNAGLIRGGYHFARPGSSSGAAQAEYFLAHGGGWTGDGITLPGMLDLEAGCSGLSASAMVSWIQDFGETYKASTGRYPMIYTTTSWWSSCTGNNGGFGDYPLVLARWASSPGELPNGWSVHSFWQNADTYEYGGDSEIWNGSQENLVKFASQ

SEQ ID NO: 32 - protein, 202 aa, Ascobolus stictoideus
AVPGFDISHYQSTVNFADAYSSGARFVIIKATEGTTYKDPKFSSHYTGATNAGLIRGGYHFARPASSTGAVQAQYFVSNGGGWSGDGLTLPGMLDLEGDCAGLSQAGMVSWITSFVNKYKALTTRYPMIYTTNSWWNTCTGNSQAFSANCPLVIARYNSVVGTIPGGWPYYTIWQFNDAYSYGGDSDTFNGAYSQLVKLATG

SEQ ID NO: 33 - protein, 207 aa, Coniochaeta sp
TVQGFDISHYQPTVNYAGAYNAGARFVIIKATEGTTYTDPSFSTHYNGATKAGLIRGGYHFAHPGVTTGAAEANFFLAHGGGWSGDGITLPGMLDLESEGSNPQCWGLSTSGMVAWIKSFSDRYHTVTGRYPMLYTNPSWWSTCTGNSNAFVNTNPLVLARYASAPGTIPGGWPYQTIWQNSDSYSYGGDSDIFNGNLASLQKLATG

SEQ ID NO: 34 - protein, 202 aa, Daldinia fissa
AVPGFDISHYQSSVDFGAAYSSGARFVIIKATEGTTYQDPKFSSHYAGATNAGLIRGGYHFARPASSSGAAQATFFLAHGGGWSGDGITLPGMLDLEGDCAGLSTSAMVSWIRDFSDTYHGKTGRYPLLYTNPSWWSSCTGGSSAFVNTNPLVLARYASSPGALPGGWPYYTIWQFNDAYKYGGDSDTFNGDLTQLKKLASG

SEQ ID NO: 35 - protein, 201 aa, Rosellinia sp
AVQGFDISHYQSSVNFAGAYSAGARFVIIKATEGTSYIDPKFSSHYIGATNAGLIRGGYHFAHLGSSSGAAQANYFLAHGGGWSGDGITLPGMLDLEGDCVLSASGAVAWIKDFSDTYHSKTGVYPLLYTNPSWWSSCTGNSNAFVNTNPLVLARYSSSAGTPPGGWPYYTIWQYNDAYAYGGDSDVFNGDMAGLLRLAKG

SEQ ID NO: 36 - protein, 202 aa, Ascobolus sp. ZY179
AVPGFDISHWQSSVNFASAYSSGARFVIIKATEGTTYKDPKFSSHYTGATKAGFIRGGYHFARPASSTGAAQAQFFASNGGGWSGDGITLPGMLDLEGDCAGLSQSGMVSWISSFVNKYRSLTGRYPMIYTTNSWWVTCTGNSKAFSSNCPLVIARYNSVVGTIPGGWPYYTIWQYNDAYKYGGDSDTFNGAYSQLVKLATG

SEQ ID NO: 37 - protein, 206 aa, Curreya sp. XZ2623
TVPGFDISHYQGTVNFAGAYSSGARFVIIKATEGTTYTDPNFSNNYVGATNAKFIRGAYHFARPDGGSGSTQAQFFHSHGGGWSGDGITLPGMLDIEYGPTSTCYGLSTSAMVTWITDFVNEYHALTGRYPLIYTTNDWWNTCTGNTNKFSTTCPLVLARYSSSVGTIPGGWPFQTIWQFNDNYAYGGDSDTFNGDLAGLKKLATG

SEQ ID NO: 38 - protein, 202 aa, Coniothyrium sp
AVQGFDISHYQASVNFAAAYSGGLRFVYIKATEGTTYQDPAFSSHYSGATSAGFIRGGYHFARPASSTGAAQASYFVAHGGGWSNDGITLPGMLDLEGDCAGLSTASMVSWISSFSNQYHSLTGRWPVIYTTNSWWTTCTGNSAAFNANSPLMLARWGSTAGTIPGGWPYYTIWQYKDSNTYGGDSDVFNGDATQLKKLATG

SEQ ID NO: 39 - protein, 202 aa, Hypoxylon sp
AVPGFDISHYQSNVDFGAAYSSGARFVIIKATEGTTYQDPSFSTHYTGATKAGLIRGSYHFARPGSSSGAAQATYFLAHGGGWSGDGITLPGMLDLEGDCAGLSTNAMVAWIRDFSDTYHGRTGRYPLLYTNPSWWSGCAGGSAAFVGTNPLVLARYAGSPGALPGGWPYYTIWQFDDAYKYGGDSDTFNGDLTQLKKLASG

SEQ ID NO: 40 - protein, 202 aa, Xylariaceae sp. 1653h
AVPGFDISHYQSSVDFAAAYSAGARFVIIKATEGTTYIDPSFSSHYTGATNAGLIRGGYHFAHPGSSSGATQANYFLAHGGGWSGDGITLPGMLDLEGDCAGLSTSAMVSWIKDFSNAYHSKTGRYPLLYTNPSWWSSCTGSSSAFVNTNPLVLARYSSSAGTPPGGWPYYTIWQFNDAYKYGGDSDTFNGEYASLQKLATG

SEQ ID NO: 41 - protein, 202 aa, Hypoxylon sp
AVPGFDISHYQSSVDFAAAYNSGARFVIIKATEGTTYIDPSFSSHYTGATKAGFIRGGYHFAHPGSSSGAAQANYFLAHGGGWSGDGITFPGMLDLEGDCAGLSTSAMVSWIKDFSDTYHSKTGRYPLLYTNPSWWSSCTGDSSAFVNTNPLVLARYSSSAGTPPGGWPYYTIWQFNDAYKYGGDSDTFNGNYASLQKLATG

SEQ ID NO: 42 - protein, 206 aa, Yunnania penicillata
DVDGFDISHYQETVDYAGAYGAGARFVIIKATEGTNYIDSSFNTHYAGATDAGLIRGGYHFAHPGETTGAEQADYFIAHGGNWSNDGITLPGMLDLEGEGSTTCWDLSAADMVAWIKAFSDRYQEVTSRYPLLYTNPSWWSECTGNSDAFVDTNPLVLARYASSPGEIPGGWPAQTIWQNSDSYSFGGDSDIFNGDEAGLKKLASG

SEQ ID NO: 43 - protein, 207 aa, Engyodontium album
RVQGFDISHYQPSVDFNAAYADGARFVIIKATEGTTYKDPKFSQHYIGATNAGFIRGGYHFAQPASSSGAAQADYFLKNGGGWSSDGITLPGMLDMEYNPNGSACYGLSQASMRNWINDFVNTYHSRTGVYPLLYTTTSWWKTCTGNTAMFADKCPLVIARYNSVVGELPAGWSFWTIWQYNDHYKHGGDSDAFNGDYSQLQRIARG

SEQ ID NO: 44 - protein, 208 aa, Metapochonia bulbillosa
TVAGFDISNYQPTVDFKKAYADGARFVIIKATEGTTYTDPSFSSHYTGATQAGLIRGGYHFAHPGSGTGAAQANYFLAHGGGWSKDGITLPGMIDLEYNPSGATCYGLSASGMVSWISDFVETYHSKTGVYPLIYTSTSWWNQCTGSSTAFGNKCPLVVARYASSVGALPAGWGFQTIWQNSDKSPWGGDNDIFNGSLDQLKRIANAS

SEQ ID NO: 45 - protein, 215 aa, Hamigera paravellanea
APLEARAGSVQGFDISHYQAKVDFAAAYRSGARFVIIKATEGTTYTDPAFSSHYTSATNAGFIRGGYHFAHPDSSSGAAQATYFLAHGGGWSGDGITLPGMLDLEYNPSGATCYGLSDAAMVAWIQDFVDTYHARTGRYPMIYTTADWWNTCTGNSSKFSQTCPLVLARYASSVGTVPGGWGYQTIWQNSDSYAYGGDSDIFNGDETQLKKLASG

SEQ ID NO: 46 - protein, 217 aa, Metarhizium iadini
SPVELEQRAASVKGFDISGYQPNVDFNKAYADGARFVIIKATEGTTYIDKTFSKHYTGATKAKLIRGAYHFAHPGQNKASAEADFFVQHGGNWSKDAITLPGMVDLESEKGHPPCWGLSHSAMVAWISEFVAAYHKKTTRYPMLYTNPSWWSACTGNSKAFKDTCPLVLARYASSPGAIPGGWPAQTIWQNSDKSPWGGDSDMFNGDLAQLKKLATG

SEQ ID NO: 47 - protein, 214 aa, Thermoascus aurantiacus
ELDKRARGVQGFDISHYQPNVDFKGAYNSGARFVIIKATEGTTYKDPAFSKHYIGATEAGLIRGGYHFAHPDKSSGAAQANFFLAHGGGWSGDGITLPGMVDLEYNPSGDACYGLSDSQMVSWIRDFVNTYHAHTGRYPMIYTTADWWKRCTGDSHAFSTTCPLVLARYNSSPGTVPGGWPYHTIWQNSDKYRFGGDSDIFNGDLAGLKRLAKG

SEQ ID NO: 48 - protein, 208 aa, Clonostachys rossmaniae
AVPGFDISGWQKSTDFAKSYANGDRFVYIKATEGTTFKNPLFSKQYTGATNARLIRGAYHFAQPASSSGASQARFFVANGGGWSNDGITLPGAVDMEYNPSGATCYGLSKTAMVNWIEDFVSTYQALTGRWPVVYTTLDWWTQCTGNSAKFGDRCPLWVARYASAVGQIPAGWSFHTIWQYNAKYPEGGDSDIFNGDETRLKALASGA

SEQ ID NO: 49 - protein, 203 aa, Simplicillium obclavatum
APKGIDVSHWQGSINWGAVKANGIEWAYIKATESTNYKDPNFNANYVGATNAGLIRGAYHFARPGDSSGAAQANYFASNGGGWSADGITLPGAVDLEAGCSGLSQSAMTAWIKDFSNTYHARTGRYPAIYTTTSWWKQCTGNASGFQNNNPLWIARWASSVGELPAGYSYHTFWQYADHGPNPGDQDVFNGDSAGLKRMAKGS

SEQ ID NO: 50 - protein, 216 aa, Aspergillus inflatus
APLEARANTVQGFDISSFQPNVDFAAAYKAGARFVMMKATQNTNYVDKTFNAHYEGATKAGLIRGGYHFAIPNGPSGAAQAEYFLAHGGGWSDDGKTLPGMIDLEYNPYGQTCYDLSAAKMVDWIKDFSNTYHAKTKRYPMIYTTANWWKECTGDSKEFSQTNPLVLARYSSSAGTVPGGWPAYSFWQNADKYKFGGDSDIWNGSEDNLKKFAKGA

SEQ ID NO: 51 - protein, 207 aa, Paracremonium inflatum
KVLGFDISHYQATVDFNAAKDAGARFVIIKATEGTTYKDPAFSKHYTGATKAGLIRGGYHFAQPASSSGAAQATFFLAHGGGWSSDGITLPGMLDLEYNPSGSTCYGLSQSSMVQWISDFIDTYHSKTGRYPLIYTSTSWWKTCTGNSSKFAANCPLVVARYSSSVGELPAGWTYYTIWQNSDSYKYGGDSDIFNGDESQLQKLAKG

SEQ ID NO: 52 - protein, 208 aa, Westerdykella sp
AVSGMDISHYQGTNYNFAGAYSSGARFVIIKATEGTTYTDPQFSANYIAATNAGFIRGGYHFARPADSTGAAQAKYFVSHGGGWSSDGITLPGMLDLEYGSSSACHGLSVSAMNTWIASFINQYRSLTGAYPMIYTTADWWKTCTGDSQAWNTKCPLVLARYSSSVGTIPGGWPYQTIWQFNDSYKYGGDSDTFNGDLAGLKRLAKGS

SEQ ID NO: 53 - protein, 207 aa, Stropharia semiglobata
LTYAVDSSTLVSVATYTKAKSQGFTKAIIRGYQEACGSGGAVDPNFVQTYKNARAAGYTDIDMYWFPCNGSTHNCKSYATQIAAIAATFSANSMKIGRIWIDIEKDAAVCNNWNYGTAGNLSQAKALISAIKASGFVYGIYSSPGEWGNIFGSTSVVVDNSAPLWFATWNNVQTLTMGTKFGGWTSAMGHQYTDVSASGQFDLSVFA

SEQ ID NO: 54 - protein, 208 aa, Gelasinospora cratophora
TVQGFDISHYQSSVNFAGAYSSGARFVIIKATEGTTYIDSSFSSHYTGATSAGLIRGGYHFAHPDSSTGAAQADYFLAHGGGWSADGITLPGMIDLESVSGKATCFGLSTSAMVSWIKSFSDRYYAKTGRYPMIYTNYSWWNQCTGNSASFAATNPLVLARWSSTVGTLPGGWSVQTIWQNADTYTYGGDSDVFNGSLDRLKALAKGS

SEQ ID NO: 55 - protein, 207 aa, Flammulina velutipes
RLNGIDVSGYQPNVNWATVKANGVSFAYIKATEGTTYTNPSFSSQYTGATKAGLIRGSYHFAHPSSSTGAAQARYFVAHGGGWSGDGITLPGALDIEYNPSGATCYGLSTSSMVNWIADFSNTYHSLTGRYPVIYTTADWWRTCTGNSASFANNSPLWIARYASTIGTLPAGWSYATFWQYADSGSNPGDQDYFNGDAAGLKRLATS

SEQ ID NO: 56 - protein, 207 aa, Deconica coprophila
LVHAVDSSSLVSTATFSKAKSEGFTKAVIRGYQEACGSGGRVDPNFVQTYKNARAAGITNIDTYWYPCNGSGNSCKSYAKQIAGISATFNAHSMKIGRIWIDIEKDSICNNWNYGTSGNRDHAKKLITAIKNSGFKYGIYSSPGEWSTIFGSESFDLDSGAPLWFATWNNVQTLTLGTHFGGWTSAHGHQYTDKSASGQFDLNVFSS

SEQ ID NO: 57 - protein, 208 aa, Rhizomucor pusillus
YETGVDVSALTSTSAWSCAKKLGYDHAIVRCYIEAYGGNPGGKIDSNCFQNYKNAKAGGFTSVDIYMFPCTGRSTCKSPAAQVKEVVDYVGSNKMTVGRLWLDVEIDPSANNWPSASSARSTLKSFKSALDSTGWKYGIYSSASQWSQITGSSSWELDSSLPLWYAHYDASLSFSDFSPFGGWTKPTIKQYAGSVSFCSAGWDKNYYG

SEQ ID NO: 58 - protein, 207 aa, Stropharia semiglobata
LVYGVDSSTLVSTATYSKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTKAVGHQYTDVSASGQFDLNVFAN

SEQ ID NO: 59 - protein, 207 aa, Stropharia semiglobata
LVYGVDSSTLVSTATYKKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTTAAGHQYTDVSSSGQFDLNVFAN

SEQ ID NO: 60 - protein, 207 aa, Myceliophthora fergusii
AVQGFDISHWQSSVDFKAAYNSGARFVIIKATEGTSFIDPKFSSHYTGATNAGFIRGAYHFAHPGQSSGEAQADYFLAHGGGWTPDGITLPGMLDLEAYNAGECWGLSQSAMVAWIKAFSDRYHARTGVYPMLYTNLSWWKTCTGNSKAFVNTNPLVLARWASSPGEIPGGWPWQTIWQNSDSYRYGGDSDIFNGDMNQLRRLATAA

SEQ ID NO: 61 - protein, 204 aa, Mortierella alpina
ALPKGIDVSHWQGDVNWNSVKAAGIEFVYIKATESINYIDSKFDANYVGATNAGLIRGGYHFARPAASSGAVQANYFLANGGGWSSDGITLPGALDLEAGCSGLSQAAMTAWVRDFSDTYHARTGRYPVIYTTTSWWKQCTGNASGFQNNNPLWIARWASSAGELPAGYAFHTFWQYADKGPNPGDQDYFNGDSAGLRRFAKGS

SEQ ID NO: 62 - protein, 216 aa, Penicillium atrovenetum
TPLESRASGVQGFDISSYQGTVDFAGAYAAGARFVMIKATEGTTYTDKTFSSHYEGASSAGLIRGGYHFAHPDSSSGAKQAEYFLAHGGGWSNDGKTLPGMLDIEYNPSGATCYGISKSAMVAWVKDFGETYKGKTGRYPMIYTTADWWNTCTGGSTAFSKDYPLVLARYSSSVGTIPGGWPYQSFWQNSDKYTYGGDSDLWNGSEASLKTFAKGA

SEQ ID NO: 63 - protein, 245 aa, Trichophaea saccata
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 64 - protein, 249 aa, Chaetomium thermophilum
PASAYAITGDNVNCRSGPGTSYAVKKVYKKGTDVKISCQTTGTNINGNNLWDKTSDGCYVSDYYVKTGSNGYVTSKCSSSGGSTCAAPKSNQATVDLIAEFEGFRANIYTDAAGYATVGYGHKCQKAKCAEVKYKIPLSKADGKKLLADDMRSFEVCITNMLNSKAKLNYNQFGALVSWSFNVGCGAAKSSTLIKRLNNGENVNKVLSEELPKWNKAGGKVLQGLVRRRAAEVALAKKSGSSQALPVKC

SEQ ID NO: 65 - protein, 248 aa, Trichoderma harzianum
YPITGDVVNCRTGPGTSYAIKKSYKKNQDISISCQTAGTSVNGNSIWDKTADGCYVADYYVKTGSSGYVTKKCTASSGGGSSSSYCKTINSAGVDLIAKWEGFVASPKPDPIGLPTVGYGHLCQQKNCREVKYKFPLTKTTAKELLLDDLPKYTKCLADYLNDKPKLNANQWAALTSWVFNVGCGNAKTSTLVKRLNNGEAANTVAAEELPKWRMAGGKVLPGLEARRKDEVKLFKTASSKQAYPKCQ

SEQ ID NO: 66 - protein, 245 aa, Trichophaea minuta
YPAKVDLRCRSSPSTSASVVRTYSKGSEIQISCQTTGTSVEGSNVWDKTQHGCYVADYYVKTGHSGIFTTKCGSSSGGGSCKPPPINAATVALIKEFEGFVAKPAPDPIGLPTVGYGHLCKTKGCKEVPYSFPLTQTTATKLLQSDIKTFTSCVSNYVKDSVKLNDNQFGALSSWAFNVGCGNIQTSSLIKRLNAGENPNTVAAQELPKWKYAGGKVLPGLVRRRKAEVALFKKPSSVQAHPPKC

SEQ ID NO: 67 - protein, 249 aa, Chaetomium sp. ZY287
YKISGSSVNCRSGPGTNYPVKKTYANGDEVTISCQTTGTNVEGNNIWDKTQHGCYVADKYVKTGKDGFVTKKCGSSGGGGGGKTCKAPKSNAATVDLIASFEGFRANIYTDATGHPTVGYGHMCTKSRCAEVKYKIPLSKADGKKLLADDMAKFEKCIKEMLNSKAKLNLNQYGALVSWSFNVGCGAAKGSQLVSRLNKGENPNTVLSNELPKWVHGNGKVLPGLVRRRNAEIALAKKSGSGAALPVKC

SEQ ID NO: 68 - protein, 245 aa, Mortierella sp. ZY002
YPITGADALHCRSGPGTSYPIQKTLRPPQDIKIQCQEPGTVVNGVSLWDKTQFGCYVSDYYVKTGTGNYVAPRCNSGGSSSACTGLNDAGINLIKEFEGFVPRPAPDPIGLPTVGYGHLCQTKGCGEVKYSFPLTTATATALLKDDLPKYTSCLAKALNGKPKLNKNQWAALASWTFNVGCGNMKSSSLITRLNAGQNPNTVATEELPKWKLAGGKVLPGLVRRRAAEVKLFKTANSSQGYPKCA

SEQ ID NO: 69 - protein, 247 aa, Metarhizium sp. XZ2431
YPVSADSLNCRAEPNTSSAIKTTYKKGEDVKISCQTEGPSINGNTIWDKTQDGCYVADYYVKTGSSGYVTGKCGGSSPPSGSGFCKTVNKAGLDLITKWEGFVSSPRGDPIGLPTVGYGHLCQKKGCAEVKYKFPLTKATALQLLNDDLPKYTGCLGKLLNSKVKLNDNQWAALTSWVFNVGCGNAQSSSLVRRLNNGENPNTVAPSELPKWKMAGGKVLEGLVKRRADEVRLFKVSSSKGAFPKCQ

SEQ ID NO: 70 - protein, 250 aa, Geomyces auratus
AFPITGSTVNCRTGPGTSHGVKTSYKKGHEVTVSCQTGGTSVNGNSIWDKTSDGCYVADYYVKTGSSGYVKPKCGSSSGGGGGSSCGAPKSNAATVNLIAEFEGFVSHVYTDATGHPTVGYGHLCSNSKCSGIGYSIPISKANAKKLLAKDMAIAEKCITAMINKSRTLNLNQYGALVSWAFNEGCGAAKSSTLIKRINNGEKPSTVIPQELPKWVYGGSSVLPGLVRRRNAEIALAKKATSSKALPAHC

SEQ ID NO: 71 - protein, 240 aa, Ilyonectria rufa
YKITGDNVNCRSGPGTSYSVKRSFKKGTDVTLSCQTTGENVLGTSIWDKTSYGCYVSDYYVKTGSSGFVVKKCGTCGAPKSNAATVNLISDFEGFRANIYKDAAGYPTVGYGHLCSNSRCTDVPYSIPLSKANGKNLLATDMTKFEKCITAMVSSSVTLNKNQYGALVSWAFNMGCGATKTSTLIKRLNQGQNVNTVLSTELPKWVYAGGKKLNGLVRRRNAEIALAKKKTTEKALPNKC

SEQ ID NO: 72 - protein, 188 aa, Nocardiopsis sp. NRRL 18262
ADIIGGLAYTMGGRCSVGFAATNAAGQPGFVTAGHCGRVGTQVTIGNGRGVFEQSVFPGNDAAFVRGTSNFTLTNLVSRYNTGGYAAVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 73 - protein, 17 aa, Nocardiopsis alba
ADIIGGLAYTMGGRCSV

SEQ ID NO: 74 - protein, 314 aa, Bacillus horneckiae
EVTATPSTQTPWGIKSIYNDQSITKTTGGSGIKVAVLDTGVHTGHIDLAGSSEQCKDFTQSNPLVNGSCTDRQGHGTHVAGTVLAHGGSDGQGVYGVAPQAKLWAYKVLGDNGSGYSDDIAAAIRHVADEASRTGSKVVINMSLGSSGKDSLIASAVDYAYGKGVLIVAAAGNSGSGSNTIGYPAALVNAVAVAALENVQQNGTYRVANFSSRGNPATAGDFRIQERDVEVSAPGASVESTWYNGGYNTISGTSMATPHVAGLAAKIWSSNSSLSHSQLRTELQNRAKVYDIKGGIGAGTGDDYASGFGYPRVK

SEQ ID NO: 75 - protein, 311 aa, Bacillus sp. [sequence omitted]

SEQ ID NO: 76 - protein, 311 aa, Artificial Sequence
  Protein Engineered variant
AVPSTQTPWGIKSIYNDQSITKTTGGKGIKVAVLDTGVYTSHLDLAGSAEQCKDFTQSNPLVDGSCTDRQGHGTHVAGTVLAHGGSNGQGVYGVAPQAKLWAYKVLGDKGEGYSDDIAAAIRHVADEASRTGSKVVINMSLGSSAKDSLIASAVDYAYGKGVLIVAAAGNEGPKPNTIGYPAGFVNAVAVAALENVQEKGTYRVADFSSRGNPATAGDYIIQERDIEVSAPGASVESTWYTGGYNTISGTSMATPHVAGLAAKIWSANTSLSHSQLRTELQNRAKVYDIKGGIGAGPGDDYASGFGYPRVK

SEQ ID NO: 77 - protein, 307 aa, Trichoderma reesei
MRPSGFLAVLSTALVGYAAAKDAPVVIDNPQGVEFKASLPKKPFFAGAALVGNVKGSVSATAGPAGKGVRFRVQFENLPKTGGPFLYHIHEEPAVNGNCTSTLAHLDPYHRGETPACNASEPQTCQVGDLSGKYGKITSDPFVAEYFDLYTSLQPGSPAFFGNRSIVIHYANKTRLTCANFAKLPVPAATTVPSTTGAHHTAIHGPLATGGAQHNLTGSHNTTGHHNFTSPHRTTGAASPSDVYTLTSVVTVPGSPTTSSSLTTTDDAGSGGASATSSVVPFPGAASGNRVSLSLLSGVVAAIFFSL

SEQ ID NO: 78 - protein, 206 aa, Aspergillus versicolor
MNLKFLPAAVLGLLISTTAAQDNNADTTDTDTNAPVITDNEPLSFHHASLLKKENTTVYGAITITTRRESAAVQVDVSIGGIPEGEYLNYHIHAARVPDDGNCYLTGAHLDPHGRGQEPPCTITAPQTCEVGDLSGKHGPAWAPAGEEFRATYSDFFLANTPGDDAYYGDLSWVVHGSNGDRLTCGNFDGFSGGGFGNWGGQKSKA

SEQ ID NO: 79 - protein, 154 aa, Aspergillus deflectus
MAAVFAIANITGIVSGIVSFSQASPGTPVSVGGVLQGLAPGLHGFHVHESPISNGDCATAGSHFNPFHVNHGGPTAEVRHVGDLGNIQADDNGFSILNIQDTHIQLSGEQNILGLSVVIHADPDDLGLGGYPDSLTTGHAGARVACADIVVPGV

SEQ ID NO: 80 - protein, 188 aa, Aspergillus egyptiacus
MYFKVFTGAFLGLVLAVSAQDSSTHAPVITNNMPISLYRATLLQKDNTTVYGGIFIGSQGSESASQIDIFLGGIPEGEHLNYHIHQYPVPENGNCYQTGAHLDPYGRGQTPPCDITAPHTCEVGDISGKHGPAFAPPGEVFSASFPDFFLSNNDDKPAFFGNRSFVVHGSGGERLTCGNFEEVDISND

SEQ ID NO: 81 - DNA, 1049 bp, Trichoderma reesei
  gene 1..1049
atgcggccgtctgggttcctggcggtcctttccaccgccctcgttggctatgccgccgcgaaagacgctcctgttgtcattgacaatcctcaaggcgtcgagttcaaggccagtctgcccaagaagccctttttcgccggcgctgcgctggtgggcaatgtcaagggatcggtctctgctacggctggtcctgctggcaagggcgtccggttcagagtccagtttgaaaacttgcccaagacgggcggtcctttttgtgagtcctcttcttgctctcgactactttctgtgtttcatctcttgtctccggtctcacttcatctttccggtcaatggctatgtgtgtgcgccctcaaagtagctaaccaatgcccgtatgcagtgtaccacatccacgaggagccggccgtcaatggcaactgtacctcaacactggcccatctggacccgtaccatcgcggcgagacacctgcctgcaatgcttccgagccccagacgtgccaggtcggtgatctgagcggaaagtacggcaaaatcacgtcggatcccttcgtggccgagtactttgacctctacacgtctctccagcccggcagcccagccttcttcggcaaccgatcaatcgtgatccattacgcaaacaagacgcggctgacgtgcgccaactttgccaagctgccggtgcccgccgcaacgacggtccccagcaccaccggtgctcatcacactgccattcatgggcccctggctaccggtggcgctcaacataacctcaccggctctcacaacaccacaggccaccacaacttcacgagcccccatcgcaccacaggcgctgcctccccctcagacgtctacaccctcaccagcgtcgtcacggtccctggcagtcctaccacctcgtcgagcctcaccaccaccgacgacgctgggagtggtggtgccagcgccacgtcatctgttgttccctttccgggtgcagcatcgggaaacagagtgtccctttccctgctgagcggtgttgtagccgccatcttcttctccctgtg

(19) United States
(12) Patent Application Publication
(10) Pub. No.: US 2024/0115669 A1
(43) Pub. Date: Apr. 11, 2024

(54) METHODS FOR REDUCING PATHOGENIC E COLI BY SELECTIVE FEED ADDITIVE INTERVENTION COMPRISING ENZYMES SUCH AS MURAMIDASE

(71) Applicants: DSM IP Assets B.V., Heerlen (NL); NOVOZYMES A/S, Bagsvaerd (DK)

(72) Inventors: Joshua CLAYPOOL, Kaiseraugst (CH); Kevin FREEMAN, Kaiseraugst (CH); Ghislain SCHYNS, Kaiseraugst (CH)

(21) Appl. No.: 18/263,818

(22) PCT Filed: Feb. 15, 2022
(86) PCT No.: PCT/EP2022/053673

§ 371 Date: Aug. 1, 2023

Related U.S. Application Data
(60) Provisional application No. 63/149,812, filed on Feb. 16, 2021.

(51) Int. Cl. A61K 38/48; A61K 9/00; A61K 38/44; A61K 38/47; A61P 31/04; C12Q 1/689

(52) U.S. Cl. CPC A61K 38/48; A61K 9/0056; A61K 38/44; A61K 38/446; A61K 38/47; A61P 31/04; C12Q 1/689; C12Y 111/01006; C12Y 115/01001; C12Y 302/01017; C12Q 2600/156

(57) ABSTRACT

The present disclosure relates to methods of modulating level of E. coli present in the gastrointestinal tract of an animal. Such modulation includes, for example, modulating the level of LEE and non-LEE pathogenic genes in the microbiome of the host animal. The present disclosure further relates to methods of modulating the virulence of pathogenic E. coli in the gastrointestinal tract of an animal by reducing the population of B. thetaiotaomicron in the gut microbiome of the animal.

### TECHNICAL FIELD

The invention pertains to the reduction of E. coli pathogenesis The invention pertains to a method for improving the health of production animals by reducing the population of E. coli pathogens in the gastrointestinal tract of the animals. More specifically, the invention pertains to methods for reducing the population of locus for enterocyte effacement (LEE) genes and the non-LEE pathogenic genes of pathogenic E. coli strains in the microbiome of their host animal. The invention also pertains to the reduction of the population of Bacteroides thetaiotaomicron in the microbiome of their host animal.

### BACKGROUND INFORMATION

Escherichia coli is an extremely versatile microorganism. In addition to being a member of the normal intestinal flora, strains of E. coli also cause bladder infections, meningitis, and diarrhea. Diarrheagenic E. coli include at least five types of E. coli, which cause various symptoms ranging from cholera-like diarrhea to extreme colitis. Each type of diarrheagenic E. coli possesses a particular set of virulence factors, including adhesins, invasins, and/or toxins, which are responsible for causing a specific type of diarrhea.

Enteropathogenic E. coli (EPEC), is a predominant cause of infantile diarrhea worldwide. EPEC disease is characterized by watery diarrhea of varying severity, with vomiting and fever often accompanying the fluid loss. In addition to isolated outbreaks in daycares and nurseries in developed countries, EPEC poses a major endemic health threat to young children (<6 months) in developing countries.

Enterohemorragic E. coli (EHEC), also called Shiga toxin producing E. coli (STEC), causes a more severe diarrhea than EPEC (enteric colitis) and in approximately 10% of cases, this disease progresses to an often fatal kidney disease, hemolytic uremic syndrome (HUS). EHEC O157:H7 is the most common serotype in Canada and the United States, and is associated with food and water poisoning (Perna et al., 2001, Nature 409: 529-533). Other serotypes of EHEC also cause significant problems worldwide. EH EC colonizes cattle and causes A/E lesions, but does not cause disease in adult animals, and instead sheds organisms into the environment. This however causes serious health problems as a relatively few EHEC are necessary to infect humans.

Unlike other E. coli diarrheas, such as enterotoxigenic E. coli, diarrhea caused by EHEC and EPEC is not mediated by a toxin. Instead, EPEC and EHEC bind to intestinal surfaces (EPEC the small bowel, EHEC the large bowel) and cause a characteristic histological lesion, called the attaching and effacing (A/E) lesion (Tauschek, et. al. 2002, Mol. Microbiol 44; 1533-1550.). A/E lesions are marked by dissolution of the intestinal brush border surface and loss of epithelial microvilli (effacement) at the sites of bacterial attachment. Once bound, bacteria reside upon cup-like projections or pedestals. Underlying this pedestal in the epithelial cell are several cytoskeletal components, including actin and actin associated cytoskeletal proteins. Formation of A/E lesions and actin-rich pedestals beneath attaching bacteria is the histopathological hallmark of A/E pathogens (Nataro, et. al., 1998, Clin Microbiol Rev 11: 142-201, and Frankel et al., 1998 Mol Microbiol 30: 911-921).

EPEC and EHEC belong to a family of A/E pathogens, including several EPEC-like animal pathogens that cause disease in rabbits (REPEC), pigs (PEPEC), and mice (Citrobacter rodentium). These pathogens contain pathogenicity islands (PAIs) that encode specialized secretion systems and secreted virulence factors critical for disease. The genes required for the formation of A/E lesions are thought to be clustered together in a single chromosomal pathogenicity island known as the locus for enterocyte effacement (LEE), which includes regulatory elements, a type III secretion system (TTSS), secreted effector proteins, and their cognate chaperones (Elliott et al., 1998, Mol Microbiol 28: 1-4. Perna, et al., 1998, Infect Immun 66: 3810-3817. Zhu, et al., 2001, Infect Immun 69: 2107-2115; Deng et al., 2001, Infect Immun 69: 6323-6335.

The LEE contains 41 genes, making it one of the more complex PAIs. The main function of the LEE TTSS is to deliver effectors into host tells, where they subvert host cell functions and mediate disease. Five LEE-encoded effectors (Tir, EspG, EspF, Map, and EspH) have been identified. Tir (for translocated intimin receptor) is translocated into host cells where it binds host cytoskeletal and signaling proteins and initiates actin polymerization at the site of bacterial attachment, resulting in formation of actin pedestal structures underneath adherent bacteria, which directly interact with the extracellular loop of Tir via the bacterial outer membrane protein intimin. CesT plays a role as a chaperone for Tir stability and secretion.

Four other LEE-encoded TTSS-translocated effectors have been characterized in A/E pathogens: EspH enhances elongation of actin pedestals; EspF plays a role in disassembly of tight junctions between intestinal epithelial cells; EspG is related to the Shigella microtubule-binding effector VirA; and Map localizes to mitochondria, but also has a role in actin dynamics. Ler (for LEE encoded regulator) is the only LEE encoded regulator identified.

Avian Pathogenic E. coli (APEC), the etiological agent of extra-intestinal infections in birds, is a pathotype that belongs to the ExPEC group. Extraintestinal infections caused by APEC are known as colibacillosis and characterized by fibrinous lesions around visceral organs, such as septicaemia, enteritis, granulomas, omphalitis, sinusitis, airsacculitis, arthritis/synovitis, peritonitis, pericarditis, perihepatitis, cellulitis, and swollen head syndrome (Kunert et al., 2015, World's Poultry Science Journal. 71; 249-258). APEC infections also lead to reduced yield, quality, and hatching of eggs. The potential for zoonotic transmission must be considered, since poultry serves as the main host for APEC and the consumption of undercooked poultry may infect humans, which can serve as a reservoir of this pathotype (Markland et al., Zoonoses and Public Health. 2015; doi: 10.1111/zph.12194).

This pathotype is the etiologic agent of extra-intestinal infections in broiler chickens and laying hens, and these are collectively known as colibacillosis. Colibacillosis is responsible for significant economic losses in many countries. It affects all cycles of production and all sectors of the poultry industry. It causes high morbidity and mortality in broiler chickens and laying hens. An E. coli strain can be designated as APEC when isolated from birds with characteristics of colibacillosis lesions and birds that were killed by this bacterium. E. coli designated as APEC must possess some virulence genes such as encoding adhesins, iron-scavenging systems, protectins, and other virulence traits. Control methods based only on predisposing factors were not effective in preventing colibacillosis.

The bacterium Bacteroides thetaiotaomicron is one of the most abundant species of the phylum Bacteroidetes, in both humans and mice, Bacteroidetes being one of the three major phyla of the intestinal microflora (Qin J et al., 2010, Nature, 464, pp. 59-65). It has been observed that, the expression of EHEC NIPH-11060424 genes involved in metabolism, colonization and virulence is modulated in response to direct contact with B. thetaiotaomicron and to soluble factors released from B. thetaiotaomicron. It was suggested that direct contact with B. thetaiotaomicron could function as a niche specific signal that primes EHEC for a more efficient interaction with the host cells thus increasing virulence potential (Iversen et al., 2015, PLoS ONE 10(2): e0118140. doi:10.1371/journal. pone.0118140).

Traditionally, reduction or elimination of pathogenic E coli strains in production animals were often focusing on bacteria supersession in the gastrointestinal tract of the animal by means of pharmaceuticals such as antibiotics. Given the increasing knowledge about microbiome and their role in the digestive system of the host animals, there is a need to identify novel, non-antibiotics ways of reducing pathogenic E. coli populations in production animals. In other words, there is a need to identify novel ways of modulating the pathogenic E. coli population in the microbiome and thus improve the health of the host animal.

### SUMMARY OF THE INVENTION

The present invention is directed to a method for reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, comprising feeding said animal with one of more of the following feed additives: N-acetyl-muramidase; protease; superoxide dismutase and/or catalase, wherein the population of exogenous LEE genes and non-LEE pathogenic genes is reduced by at least 10% lower than that of a control animal which is fed with the same diet except for said feed additives. In one embodiment, the population of exogenous LEE genes and non-LEE pathogenic genes is measured as % ratio of the combined copy numbers of LEE genes and non-LEE genes detected within the microbiome of said animal vs. the total copy number of genes detected within said microbiome. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f″) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (I″) a fragment of the polypeptide of (a″), (b″), (c″), (d″), (e″), (f″), (g″), (h″), (i″), (j″), or (k″) that has superoxide dismutase activity.

The present invention is also directed to a method for reducing the population of Bacteroides thetaiotaomicron in the gastrointestinal tract (GIT) of an animal, comprising feeding said animal with one of more of the following feed additives: N-acetyl-muramidase; protease; superoxide dismutase and/or catalase, wherein the population of Bacteroides thetaiotaomicron is reduced by at least 10% lower than that of a control animal which is fed with the same diet except for said feed additives. In one embodiment, the population of Bacteroides thetaiotaomicron is measured as % ratio of the population of Bacteroides thetaiotaomicron detected within the microbiome of said animal against the total population of microbes within said microbiome. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f″) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (I″) a fragment of the polypeptide of (a″), (b″), (c″), (d″), (e″), (f″), (g″), (h″), (i″), (j″), or (k″) that has superoxide dismutase activity.

The present invention is further directed to a method for reducing systemic inflammation and/or local inflammation of an animal caused by E. coli infection, comprising feeding said animal with one of more of the following feed additives: N-acetyl-muramidase; protease; superoxide dismutase and/or catalase, wherein the systemic inflammation and/or local inflammation of the animal is reduced by at least 10% lower than that of a control animal which are fed with the same diet except for said feed additives. In one embodiment, the reduction of inflammation is measured as % ratio of the copy number of LEE and non-LEE genes detected within the microbiome of said animal against the total copy number of genes detected within said microbiome. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f″) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (I″) a fragment of the polypeptide of (a″), (b″), (c″), (d″), (e″), (f″), (g″), (h″), (i″), (j″), or (k″) that has superoxide dismutase activity.

In some embodiments of the above inventions, the microbiome is collected from either a fecal sample of the animal or a sample collected within the GIT of the animal. In some embodiments, the gene copy number measurement is performed by RT-PCR counting, full length 16S RNA sequencing, or Metagenomic DNA sequencing. In one embodiment, the LEE genes comprise: Tir, Map, EspB, EspF, EspG, EspH, and EspZ, and the non-LEE pathogenic genes comprise: EspG2, EspJ, EspM1/2, EspT, EspW, Cif, NleA, NIeB, NIeC, NIeD, NleE, NIeF, and NleH. In one embodiment, the method of the present invention is applicable to production animal.

### BRIEF DESCRIPTION OF DRAWINGS

FIG. 1 is a graph showing the % change of the population of LEE genes and non-LEE genes in the microbiome of the broilers which was fed a diet with added protease Ronozyme ProAct and that of the control broiler group without any added Ronozyme ProAct.

FIG. 2 is a graph showing the change of relative abundance of E. coli in the GIT of the host broilers which was fed a diet with added protease Ronozyme ProAct and that of the control broiler group without any added Ronozyme ProAct.

FIG. 3 is a graph showing the % change of the population of LEE genes and non-LEE genes in the microbiome of the swine which was fed a diet with added superoxide dismutase and that of the control swine group without any added superoxide dismutase.

FIG. 4 is a graph showing the change of relative abundance of E. coli in the GIT of the host swine which was fed a diet with added superoxide dismutase and that of the control swine group without any added superoxide dismutase.

FIG. 5 is a graph showing the change of relative abundance of E. coli over time in the GIT of the host turkey which was fed a diet with added lysozyme Balancius and that of the control turkey group without any added Balancius.

### DETAILED DESCRIPTION OF THE INVENTION

The terminology used herein is for the purpose of describing particular cases only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

It is understood that terms such as “comprises,” “comprised,” “comprising,” and the like have the meaning attributed to it in U.S. Patent law; i.e., they mean “includes,” “included,” “including,” and the like and are intended to be inclusive or open ended and does not exclude additional, unrecited elements or method steps; and that terms such as “consisting essentially of” and “consists essentially of” have the meaning ascribed to them in U.S. Patent law; i.e., they allow for elements not explicitly recited, but exclude elements that are found in the prior art or that affect a basic or novel characteristic of the invention.

### Definitions

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or B; A or C; B or C; A and B; A and C; B and C; A (alone); B (alone); and C (alone).

The terms “microbiome” and “gut microbiome”, which are used interchangeably in this application, refer to microbes such as bacteria, viruses, fungi, mold, protozoa, etc. that reside in the digestive track, and is responsible for converting undigested and unabsorbed components of an animal's diet into thousands of biologically active metabolites. These metabolites interface in turn with the local and systemic physiology of the animal as well as the animal's external environment.

Method of Reducing the Population of Pathogenic E. coli Bacteria in the Microbiome of an Animal

In this invention, a method of improving the health of production animal is shown. A preferred embodiment of the method of the invention relates to a method of improving the health of a production animal by reducing the population of E. coli bacteria in the microbiome of the animal. In one embodiment, a method of the invention relates to a method of improving the health of a production animal by reducing the population of pathogenic E coli bacteria in the microbiome of the animal while making lesser or insignificant impact on the non-pathogenic E. coli. In a preferred embodiment, the selective modulation of E. coli population is achieved by reducing the population of exogenous locus for enterocyte effacement (LEE) genes and exogenous non-LEE pathogenic genes of pathogenic E. coli bacteria such as Enterohemorrhagic E. coli (EHEC), Enteropathogenic E. coli (EPEC), and Avian Pathogenic E. coli (APEC) in the microbiome of the animal. In another embodiment, the invention relates to a method of improving the health of a production animal by reducing the population of Bacteroides thetaiotaomicron in the microbiome of the animal. In a specific embodiment, the above health benefit is instigated by feeding the production animal with selective feed additives such as N-acetyl-muramidase; protease; superoxide dismutase and/or catalase, as described herein.

Enterohemorrhagic E. coli (EHEC), Enteropathogenic E. coli (EPEC), and Avian Pathogenic E. coli (APEC) are a diarrheagenic human pathogen. The hallmark of infections by these pathogenic E. coli strains is the formation of the attaching and effacing (A/E) lesion in the intestinal epithelial cells, characterized by the effacement of brush border microvilli and the intimate bacterial attachment to the enterocyte in actin-rich pedestal-like structures. The locus of enterocyte effacement (LEE) in the pathogenic E. coli genome encodes a type III protein secretion system (T3SS) that translocates multiple effector proteins into the host cell to subvert cellular functions for the benefit of the pathogen. These effectors are encoded by genes both within and outside the LEE region. In vitro cell culture infections have shown that LEE effectors are required for intimate bacterial attachment to the epithelial cells, whereas non-LEE effectors mostly play a role in modulating inflammation and cell apoptosis in the gut epithelium (Massie) et al., 2020, DOI: 10.5772/intechopen.91677).

Surprisingly, inventors of present application have identified a few selective nutritional feed additives such as the N-acetyl-muramidase; protease; superoxide dismutase and/or catalase described herein, which can significantly interfere with the growth of pathogenic E. coli such as EHEC, EPEC and APEC. It has been shown in the present invention that feeding suitable amount of above selective feed additive can help to reduce the population of LEE genes and non-LEE pathogenic genes in the microbiome of the host animal. In other words, the LEE genes of pathogenic E. coli bacteria, which responsible for forming the attaching and effacing (A/E) lesion in the gut epithelial cells of the host animal, are reduced in term of its % population within the GIT microbiome of the host animal when treated by selective nutritional additives. Furthermore, it is observed by the inventors of present application that the non-LEE pathogenic genes of pathogenic E. coli bacteria, which are responsible for modulating inflammation and cell apoptosis in the gut epithelium, are also reduced in term of its % population within the GIT microbiome of the host animal. This leads to reduced systemic and local infection of the GIT of the host animal.

It is also observed by the inventors of present application that the selective nutritional feed additives can reduce the % population of E. coli within the GIT microbiome of the host animal. Specifically, the % population of pathogenic E. coli bacteria within the GIT microbiome is reduced, possibly due to the observed reductions in the abundance of the LEE-encoded and non-LEE-encoded effectors of EHEC, EPEC and APEC.

Equally surprising, inventors of present application found that the same selective nutritional feed additives can significantly reduce the % population of Bacteroides thetaiotaomicron within the GIT microbiome. The virulence of Enterohaemorrhagic E. coli (EHEC) is reported to be coordinated with B. thetaiotaomicron, a gut commensal. Impacting this commensal can have subsequent effects on EHEC. It is known that B. thetaiotaomicron functions as a niche specific signal that primes EHEC for a more efficient interaction with the host cells and thus increasing virulence potential. Thus, reduction or removal of B. thetaiotaomicron from the GIT microbiome can diminish the interaction of EHEC with the GIT epithelial cells of the host animal and thus prevent or alleviate the pathogenicity of E. coli such as EHEC against the host animal.

On the physiology level of the animal, the selective nutritional feed additives identified in this application help to treat diarrhea and nutrient malabsorption and other poor health outcomes of animal. This is achieved by reducing the population of pathogenic E. coli bacteria and their coordinator B. thetaiotaomicron in the microbiome of the host animal and thus alleviating the pathogenicity caused by such bacteria.

Thus, a preferred embodiment of the invention relates to a method for reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, comprising the step of feeding said animal with one of more of the following feed additives: N-acetyl-muramidase; protease; superoxide dismutase and/or catalase, wherein the population of exogenous LEE genes and non-LEE pathogenic genes is reduced by at least 10% lower than that of a control animal which is fed with the same diet except for said feed additives. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f″) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (I″) a fragment of the polypeptide of (a″), (b″), (c″), (d″), (e″), (f″), (g″), (h″), (i″), (j″), or (k″) that has superoxide dismutase activity.

In one embodiment, the reduction of the population of exogenous LEE genes and non-LEE pathogenic genes is measured by the % ratio of LEE genes and non-LEE genes against the total amount of genes in the microbiome. In other words, the reduction is measured as the change of the population of pathogenic E. coli in the microbiome. In another embodiment, the reduction is measured by the % ratio of LEE genes and non-LEE genes against the copy number of an E. coli housekeeping gene. In other words, the reduction is measured as the change of the population of pathogenic E. coli in the overall E. coli population of the microbiome. In some embodiments, the reduction of the population of the % ratio of LEE genes and non-LEE genes is by at least 5%, at least 15%, at least 20%, at last 30%, at least 40%, at least 50%, at least 60%, at least 70%, at least 80%, or at least 90% lower than that of the control animal. In one embodiment, the LEE genes comprise: Tir, Map, EspB, EspF, EspG, EspH, and EspZ. In another embodiment, the non-LEE pathogenic genes comprise: EspG2, EspJ, EspM1/2, EspT, EspW, Cif, NIeA, NIeB, NIeC, NIeD, NIeE, NIeF, and NIeH.

Another preferred embodiment of the invention relates to a method of reducing the population of Bacteroides thetaiotaomicron in the gastrointestinal tract (GIT) of an animal, comprising feeding said animal with one of more of the following feed additives: N-acetyl-muramidase; protease; superoxide dismutase and/or catalase, wherein the population of Bacteroides thetaiotaomicron is reduced by at least 10% lower than that of a control animal which is fed with the same diet except for said feed additives. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f″) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (I″) a fragment of the polypeptide of (a″), (b″), (c″), (d″), (e″), (f″), (g″), (h″), (i″), (j″), or (k″) that has superoxide dismutase activity.

In some embodiments, the reduction of the population of the % ratio of Bacteroides thetaiotaomicron against the total number of microbes in the microbiome is by at least 5%, at least 15%, at least 20%, at last 30%, at least 40%, at least 50%, at least 60%, at least 70%, at least 80%, at least 90%, or at least 95% lower than that of the control animal.

Another preferred embodiment of the invention relates to a method of reducing systemic inflammation and/or local inflammation of an animal caused by E. coli infection, comprising feeding said animal with one of more of the following feed additives: N-acetyl-muramidase; protease; superoxide dismutase and/or catalase, wherein the systemic inflammation and/or local inflammation of the animal is reduced by at least 10% lower than that of a control animal which are fed with the same diet except for said feed additives. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f″) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (I″) a fragment of the polypeptide of (a″), (b″), (c″), (d″), (e″), (f″), (g″), (h″), (i″), (j″), or (k″) that has superoxide dismutase activity.

In some embodiments, the reduction of the population of the % ratio of the inflammation related non-LEE genes against the total amount of genes in the microbiome is by at least 5%, at least 15%, at least 20%, at last 30%, at least 40%, at least 50% at least 55%, or at least 60% lower than that of the control animal. In one embodiment, the inflammation related non-LEE genes comprise: NIeA, NIeB, NIeC, NIeD, NIeE, NIeF, and NIeH.

Another preferred embodiment of the invention relates to a method of reducing the population of E. coli in the gastrointestinal tract (GIT) of an animal, comprising feeding said animal with one of more of the following feed additives: N-acetyl-muramidase; protease; superoxide dismutase and/or catalase, wherein the systemic inflammation and/or local inflammation of the animal is reduced by at least 10% lower than that of a control animal which are fed with the same diet except for said feed additives. In a preferred embodiment, the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide; the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f″) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (I″) a fragment of the polypeptide of (a″), (b″), (c″), (d″), (e″), (f″), (g″), (h″), (i″), (j″), or (k″) that has superoxide dismutase activity. In a specific embodiment, said E. coli is pathogenic E. coli. In specific embodiment, the pathogenic E. coli comprises EPEC, EHEC, and APEC.

In one embodiment, the population of E. coli in the GIT of the animal is measured as % of copy number of E. coli marker genes within the microbiome of said animal against the total copy number of bacterial marker genes detected within said microbiome. In some embodiments, the reduction of E. coli population in the GIT of the animal is by at least 5%, at least 15%, at least 20%, or at last 30% lower than that of the control animal.

In one embodiment, the microbiome is collected from the fecal digesta sample of the animal. In another embodiment, the microbiome is collected from a location within the GIT of the animal. In an embodiment, the microbiome is collected from the GIT of a chicken. In some embodiments, the location is the duodenum, jejunum, ileum, cecum, or colorectum of a chicken.

Measurement of population of any genes of any microbe in the microbiome or the population of the microbiome can be conducted using any existing or future method which are suitable for the purpose. In one embodiment, such measurement is performed by metagenomic DNA sequencing. In another embodiment, the measurement is performed by RT-PCT counting. In a specific embodiment, bacterial housekeeping maker gene rpoB is used in the RT-PCT counting. In another embodiment, the measurement is performed by full length 16S RNA sequencing.

It has been observed in the present invention that above described health benefits are instigated by adding a selective feed additives to the feed of production animals. These additives are precision compounds which selectively modulate the composition and functions of the microbiome with the host animal. This selective modulation of the microbiome targets pathogenic E. coli bacteria and their coordinator B. thetaiotaomicron in the microbiome of the host animal.

In one embodiment, such feed additives are N-acetyl-muramidase; protease; superoxide dismutase and/or catalase as decribed herein. In a preferred embodiment, the feed additive comprises further feed enzymes. Feed enzymes broadly refer to exogenous enzymes in animal diets with their functions to overcome the adverse effects of antinutritional factors and improve digestion of dietary components and animal performance. Gut health enzymes refer to exogenous enzymes in animal diets with their functions to improve the health of microbiome in the GIT of the host animal. In another preferred embodiment, the feed additive comprises further gut health enzymes. It has been observed in the present invention that enzyme which are particularly effective in producing the health benefits described in this application include but are not limited to feed enzymes and gut health enzymes. Examples of feed enzyme include protease, phytases, xylanases, cellulases, mannanases, α-galactosidases, pectinases, and amylases. In a preferred embodiment, the gut health enzyme is a N-acetyl-muramidase. An N-acetyl-muramidase enzyme (sometimes also referred to as lysozyme, muramidase, or N-acetylmuramide glycanhydrolase) hydrolyzes the beta-1,4-link between N-acetylglucosamine and N-acetylmuramic acid of peptidoglycan. In a specific embodiment, the N-acetyl-muramidase is made by DSM Nutritional Products LLC in the commercial name of Balancius. In another preferred embodiment, the protease is made by DSM Nutritional Products LLC in the commercial name of Ronozyme ProAct. In another embodiment, the gut health enzyme is a superoxide dismutase or catalase.

N-acetyl-muramidase: The N-acetyl-muramidase Balancius is characterized in that it is selected from the group consisting of: (a) a polypeptide having at least 80%, e.g., at least 85%, at least 90%, at least 95%, or 100% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and/or (d) a fragment of a polypeptide of (a) or (b) having muramidase activity and having at least 90% of the length of the mature polypeptide. An enzyme referred to as N-acetyl-muramidase Balancius or lysozyme Balancius or muramidase Balancius, or N-acetylmuramide glycanhydrolase Balancius can be produced as described in Example 2 of WO 2019/121937 A1. N-acetyl-muramidase activity can be determined as described in Example 1 of WO 2019/121937 A1.

Protease: The protease Ronozyme ProAct is a serine protease obtained or obtainable from Nocardiopsis sp. In particular, it can be characterized in that it is derived from Nocardiopsis sp. NRRL 18262, and/or from Nocardiopsis alba (taxonomy based on Berge's Manual of Systematic Bacteriology, 2nd edition, 2000, Springer (preprint: Road Map to Bergey's)). It can also be characterized in that it is an acid-stable serine protease obtained or obtainable from Nocardiopsis dassonvillei subsp. dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2), as well as homologous proteases therefrom. In general, the term serine protease refers to serine peptidases and their clans as defined in the Handbook of Proteolytic Enzymes, A. J. Barrett, N. D. Rawlings, J. F. Woessner (eds), Academic Press (1998). In the 1998 version of this handbook, serine peptidases and their clans are dealt with in chapters 1-175. Serine proteases may be defined as peptidases in which the catalytic mechanism depends upon the hydroxyl group of a serine residue acting as the nucleophile that attacks the peptide bond. Examples of serine proteases for use according to the invention are proteases of Clan SA, e. g. Family S2 (Streptogrisin), e. g. Sub-family S2A (alpha-lytic protease), as defined in the above Handbook.

In addition or alternatively, the protease Ronozyme ProAct can be characterized in that it is (a) a polypeptide having a sequence identity of at least 70% to any one of SEQ ID NOs 72-76; (b) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c) a polypeptide comprising the polypeptide of (a) or (b) and a N-terminal and/or C-terminal His-tag and/or HQ-tag; (d) a polypeptide comprising the polypeptide of (a) or (b) and a N-terminal and/or C-terminal extension of up to 10 amino acids, e.g. 1, 2, 3, 4, 5, 6, 7, 8, 9 or 10 amino acids; or (e) a fragment of the polypeptide of (a) or (b) having protease activity and having at least 90% of the length of the mature polypeptide.

Protease activity can be measured using any assay, in which a substrate is employed, that includes peptide bonds relevant for the specificity of the protease in question. Examples of protease substrates are casein, and pNA-substrates, such as Suc-AAPF-pNA (available e.g. from Sigma S-7388). Another example is Protazyme AK (azurine dyed crosslinked casein prepared as tablets by Megazyme T-PRAK). Example 2 of WO 01/58276 describes suitable protease assays. A preferred assay is the Protazyme assay of Example 2D (the pH and temperature should be adjusted to the protease in question as generally described previously).

The term “protease” is defined herein as an enzyme that hydrolyses peptide bonds. It includes any enzyme belonging to the EC 3.4 enzyme group (including each of the thirteen subclasses thereof http://en.wikipedia.org/wiki/Category:EC_3.4). The EC number refers to Enzyme Nomenclature 1992 from NC-IUBMB, Academic Press, San Diego, California, including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The term “subtilases” refer to a sub-group of serine protease according to Siezen et al., Protein Engng. 4 (1991) 719-737 and Siezen et al. Protein Science 6 (1997) 501-523. Serine proteases or serine peptidases is a subgroup of proteases characterized by having a serine in the active site, which forms a covalent adduct with the substrate. Further, the subtilases (and the serine proteases) are characterized by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family.

A protease referred to herein may not only be natural or wildtype proteases, but also any mutants, variants, fragments etc. thereof exhibiting protease activity, as well as synthetic proteases, such as shuffled proteases, and consensus proteases. Such genetically engineered proteases can be prepared as is generally known in the art, e. g. by Site-directed Mutagenesis, by PCR (using a PCR fragment containing the desired mutation as one of the primers in the PCR reactions), or by Random Mutagenesis. The preparation of consensus proteins is described in e. g. EP 0 897 985.

Such non-wildtype proteases may be based on protease(s) derived from Nocardiopsis sp. NRRL 18262, and Nocardiopsis alba and have at least 60, 65, 70, 75, 80, 85, 90, or at least 95% amino acid identity but not 100% to a wildtype protease. For calculating percentage identity, any computer program known in the art can be used. Examples of such computer programs are the Clustal V algorithm (Higgins, D. G., and Sharp, P. M. (1989), Gene (Amsterdam), 73, 237-244; and the GAP program provided in the GCG version 8 program package (Program Manual for the Wisconsin Package, Version 8, Genetics Computer Group, 575 Science Drive, Madison, Wisconsin, USA 53711) (Needleman, S. B. and Wunsch, C. D., (1970), Journal of Molecular Biology, 48, 443-453. In a particular embodiment, the protease referred to herein may be both, acid-stable and thermostable. The term “thermostable” means for proteases referred to herein to have a temperature optimum is at least 50° C., 52° C., 54° C., 56° C., 58° C., 60° C., 62° C., 64° C., 66° C., ° 68 C, or at least ° 70 C.

Superoxide dismutase: The superoxide dismutase referred to herein can be characterized in that it is selected from the group consisting of: a) a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 77; b) a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 78; c) a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 79; d) a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 80; e) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 52 to 170 of SEQ ID NO: 77; f) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 55 to 136 of SEQ ID NO: 78; g) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 12 to 149 of SEQ ID NO: 79; h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acid residues 47 to 179 of SEQ ID NO: 80; i) a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the sequence of SEQ ID NO: 81; j) a polypeptide encoded by a polynucleotide that hybridizes under medium-high stringency conditions with coding sequence of SEQ ID NO: 81 or the full-length complement thereof; k) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more (e.g., several) positions and that has superoxide dismutase activity; and I) a fragment of the polypeptide of (a), (b), (c), (d), (e), (f), (g), (h), (i), (j), or (k) that has superoxide dismutase activity.

SEQ ID NO: 77 is a mature polypeptide having superoxide dismutase (SOD) activity from Trichoderman reesei comprising 307 amino acid residues wherein residues 1 to 20 make up the signal peptide and residues 52 to 170 make up the SOD. SEQ ID NO: 78 is a mature polypeptide having superoxide dismutase (SOD) activity from Aspergillus versicolor comprising 206 amino acid wherein residues 1 to 20 make up the signal peptide and residues 55 to 136 make up the SOD. SEQ ID NO: 79 is a mature polypeptide having superoxide dismutase (SOD) activity from Aspergillus deflectus comprising 154 amino acid residues wherein residues 1 to 16 make up the signal peptide and residues 12 to 149 make up the SOD. SEQ ID NO: 80 is a mature polypeptide having superoxide dismutase (SOD) activity from Aspergillus egyptiacus comprising 188 amino acid residues wherein residues 1 to 19 make up the signal peptide and residues 47 to 179 make up the SOD. SEQ ID NO: 81 is a polynucleotide sequence coding sequence of the polypeptide of SEQ ID NO: 77.

Superoxide dismutase (SOD, EC 1.15.1.1) is an enzyme that alternately catalyzes the dismutation (or partitioning) of the superoxide (02) radical into either ordinary molecular oxygen (O₂) or hydrogen peroxide (H₂O₂). Superoxide is produced as a by-product of oxygen metabolism and, if not regulated, causes many types of cell damage. Thus, SOD is an important antioxidant defense in nearly all living cells exposed to oxygen. SODs are used in the pharmaceutical, cosmetic, food, and environmental protection industries due to their excellent antioxidant properties. Historically, SODs were isolated from animal or plant sources, but the microbial sources organisms can be easily induced and cultivated on a large scale SODs naturally occur in many organisms such as plants, insects, birds, reptiles and mammals. Four types of SODs have been reported according to their metal cofactors: manganese SOD (Mn-SOD), iron SOD (Fe-SOD), copper/zinc SOD (Cu/Zn-SOD), and nickel SOD (Ni-SOD)₂. For example, mammalian (bovine) SOD and bacterial (E. coli) Mn-SOD (S5639) are commercially available from Sigma. Preferred superoxide dismutases are SODs of fungal origin. As an alternative to a superoxide dismutase, a catalase enzyme may be used, which catalase enzyme is capable of transforming H₂O₂ into H₂O and O₂.

In order to produce the health benefits described in this application, a suitable amount of enzyme is required. Such suitable amount is based on the type of animal and its stage of growth and can be determined by experiments. However, a minimal amount of enzyme (i.e. N-acetyl-muramidase, protease, superoxide dismutase and/or catalase) is required in order to obtain the health benefits. In one embodiment, the enzyme is at least 25 g/tone of the feed. In another embodiment, the enzyme is at least 50 g/tonne of the feed. An optimal range of concentration which suits best for the present invention has been determined by the inventors of the present application. In some embodiments, the enzyme is between 25-50 g/tonne, 50-100 g/tonne, 100-200 g/tonne, 200-500 g/tonne or 500-1000 g/tonne of the feed. In a preferred embodiment, the enzyme is between 50-220 g/tonne of the feed. In another embodiment, the enzyme is between 1000-100,000 IU, 5,000-100,000 IU, 5,000-50,000 IU or 10,000-50,000 IU of the feed. In one embodiment, the enzyme is a protease, wherein said protease is administered in a dosage of between 10′000 units/kg feed and 30′000 units/kg feed, for example in one of the following amounts (dosage ranges): 10′000 units/kg feed, 11′000, 12′000, 13′000, 14′000, 15′000, 16′000, 17′000, 18′000, 19′000, 20′000 units/kg feed. One protease unit (PROT) is the amount of enzyme that releases 1 μmol of p-nitroaniline from 1 mM substrate (Suc-Ala-Ala-Pro-Phe-pnA) per minute at pH 9.0 and 37° C.

In some embodiments, the invention relates to a use of feed enzymes, in particular N-acetyl-muramidase, protease, superoxide dismutase and/or catalase, for a) reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, wherein the population of exogenous LEE genes and non-LEE pathogenic genes is reduced by at least 10% lower than that of a control animal which is fed with the same diet except for said feed additives; b) reducing the population of Bacteroides thetaiotaomicron in the gastrointestinal tract (GIT) of an animal, wherein the population of Bacteroides thetaiotaomicron is reduced by at least 10% lower than that of a control animal which is fed with the same diet except for said feed additives; c) reducing the population of E. coli in the gastrointestinal tract (GIT) of an animal, wherein the systemic inflammation and/or local inflammation of the animal is reduced by at least 10% lower than that of a control animal which are fed with the same diet except for said feed additives; and/or for d) reducing systemic inflammation and/or local inflammation of an animal caused by E. coli infection, wherein the systemic inflammation and/or local inflammation of the animal is reduced by at least 10% lower than that of a control animal which are fed with the same diet except for said feed additives. In some embodiments, said use relates to the use of N-acetyl-muramidase, protease, superoxide dismutase and/or catalase for a) reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, wherein the population of exogenous LEE genes and non-LEE pathogenic genes is reduced by at least 10% lower than that of a control animal which is fed with the same diet except for said feed additives; b) reducing the population of Bacteroides thetaiotaomicron in the gastrointestinal tract (GIT) of an animal, wherein the population of Bacteroides thetaiotaomicron is reduced by at least 10% lower than that of a control animal which is fed with the same diet except for said feed additives; c) reducing the population of E. coli in the gastrointestinal tract (GIT) of an animal, wherein the systemic inflammation and/or local inflammation of the animal is reduced by at least 10% lower than that of a control animal which are fed with the same diet except for said feed additives; and/or for d) reducing systemic inflammation and/or local inflammation of an animal caused by E. coli infection, wherein the systemic inflammation and/or local inflammation of the animal is reduced by at least 10% lower than that of a control animal which are fed with the same diet except for said feed additives.

### Type of Animal

The method of the present invention is applicable to production animals in general. In one embodiment, the method of the present invention is applicable to poultry.

The above mentioned feed additives may be provided to any suitable animal. In some embodiments, the animal is monogastric. It is generally understood that a monogastric animal has a single-chambered stomach. In other embodiments, the animal is a ruminant. It is generally understood that a ruminant has a multi-chambered stomach. In some embodiments, the animal is a ruminant in the pre-ruminant phase. Examples of such ruminants in the pre-ruminant phase include nursery calves.

In some embodiments, the animal is a poultry (e.g. chicken, turkey), seafood (e.g. shrimp), sheep, cow, cattle, buffalo, bison, pig (e.g. nursery pig, grower/finisher pig), cat, dog, rabbit, goat, guinea pig, donkey, camel, horse, pigeon, ferret, gerbil, hamster, mouse, rat, bird, or human.

In some embodiments, the animal is livestock. In some embodiments, the animal is a companion animal. In some embodiments, the animal is poultry. Examples of poultry include chicken, duck, turkey, goose, quail, or Cornish game hen. In one variation, the animal is a chicken. In some embodiments, the poultry is a layer hen, a broiler chicken, or a turkey.

In other embodiments, the animal is a mammal, including, for example, a cow, a pig, a goat, a sheep, a deer, a bison, a rabbit, an alpaca, a llama, a mule, a horse, a reindeer, a water buffalo, a yak, a guinea pig, a rat, a mouse, an alpaca, a dog, or a cat. In one variation, the animal is a cow. In another variation, the animal is a pig. In another variation, the animal is a sow.

### Administration of Feed Additives

In some embodiments, administration comprises providing the feed additives described herein to an animal such that the animal may ingest the feed additives at will. In such embodiments, the animal ingests some portion of the feed additives.

The feed additives described herein may be provided to the animal on any appropriate schedule. In some embodiments, the animal is the feed additives described herein on a daily basis, on a weekly basis, on a monthly basis, on an every other day basis, for at least three days out of every week, or for at least seven days out of every month.

In some embodiments, the feed additives described herein is administered to the animal multiple times in a day. For examples, in some embodiments, the feed additives described herein is administered to the animal at least 1, 2, 3, 4, 5, 6, 7, 8, 9, or 10 times a day. In some embodiments, the nutritional composition, the feed additives described herein is administered to the animal at most 1, 2, 3, 4, 5, 6, 7, 8, 9, or 10 times a day.

In some embodiments, the feed additives described herein is administered to the animal multiple times in a day. For examples, in some embodiments, the feed additives described herein is administered to the animal at least 1, 2, 3, 4, 5, 6, 7, 8, 9, or 10 times a week. In some embodiments, the nutritional composition, the feed additives described herein is administered to the animal at most 1, 2, 3, 4, 5, 6, 7, 8, 9, or 10 times a week. In some embodiments, the feed additives described herein is administered to the animal every day, every other day, every 3 days, every 4 days, every week, every other week, or every month.

In some embodiments, the animal is the feed additives described herein during certain diet phases. For example, some animals are provided a starter diet between 0 to 14 days of age. In other embodiments, an animal is provided a grower diet between 15 to 28 days of age, between 15 to 35 days of age, or between 15 to 39 days of age. In still other embodiments, an animal is provided a finisher diet between 29 to 35 days of age, between 36 to 42 days of age, or between 40 to 46 days of age.

In certain embodiments, the feed additives described herein is provided to the animal during the starter diet phase, the grower diet phase, or the finisher diet phase, or any combinations thereof. In certain embodiments, the animal is poultry, and the poultry is provided a starter diet between 0 to 15 days of age, a grower diet between 16 to 28 days of age, and a finisher diet between 29 to 35 days of age. In other embodiments, the animal is poultry, and the poultry is provided a starter diet between 0 to 14 days of age, a grower diet between 15 to 35 days of age, and a finisher diet between 36 to 42 days of age. In still other embodiments, the animal is poultry, and the poultry is provided a starter diet between 0 to 14 days of age, a grower diet between 15 to 39 days of age, and a finisher diet between 20 to 46 days of age.

In some embodiments, the feed additives described herein is provided to the poultry during the starter diet phase, the grower diet phase, or the finisher diet phase, or any combinations thereof.

The feed additives described herein may be fed to individual animals or an animal population. For example, in one variation where the animal is poultry, the feed additives described herein may be fed to an individual poultry or a poultry population.

The feed additives described herein may be provided to an animal in any appropriate form, including, for example, in solid form, in liquid form, or a combination thereof. In certain embodiments, the feed additives described herein is a liquid, such as a syrup or a solution. In other embodiments, the feed additives described herein is a solid, such as pellets or powder. In yet other embodiments, the feed additives described herein may be fed to the animal in both liquid and solid components, such as in a mash. A solid form is typically added before or during the mixing step; and a liquid form is typically added after the pelleting step.

### EXAMPLES

### Example 1

Example 1 of the study describes the protocols and methods used for generating and analyzing the data in this invention.

### Sample Collection

Cecal digesta samples were collected at various days depending on the their species and growing schedule from both Negative Control and treatment groups (1 bird/pen and 21 replicates/treatment). Cecal samples were kept frozen at −80° C. before DNA extraction for metagenomics or solvent extraction for metabolomics analysis.

### DNA Extraction and Sequencing

Quantitative measurement of gene copies can be made through any shotgun sequencing measurement method. In this application, metagenomic DNA was extracted using MoBio Powersoil following manufacturer instructions (Qiagen, Germany). DNA was sequenced at Diversigen (TX, USA), on an Illumina HiSeq 3000 apparatus with a target depth of 5 GB per sample.

### Taxonomy Read Processing

In order to choose appropriate filtering and trimming parameters, the raw fastq files from shallow 122 shotgun sequencing were inspected using FastQC v0.11.5. Based on the quality reports, Cutadapt was used to trim the first 10 bases of each read, shorten each read to a maximum of 130 bp, and discard any read less than 120 bp long. This removed any remaining adapter fragments and eliminated regions near the end of the read where the quality dropped, confirmed by another quality report from FastQC.

### Taxonomy Analysis

Sequences read from the instrument can then be aligned using any alignment algorithm against a reference database of genes containing at a minimal, LEE and non-LEE genes. In the present application, MetaPhlan 2.0, analysis type “rel_ab_w_read_stats” was used to construct a profile of taxonomic relative abundance for each sample from the processed reads using forward reads only.

### Functional Mapping

The processed reads were mapped against an internal gene catalog specifically tailored to the chicken gut microbiome with bwa v0.7.5 using the BWA-MEM algorithm. Python scripts were used to extract a table of gene counts for each sample from the BAM files, which was used as the input for downstream analysis. Only reads that mapped in a proper pair were considered a successful hit to a gene. An internal gene catalog has been annotated using the publicly available KEGG Orthology (KO) database, and much of the metagenomics analysis discussed here is based on functional information from KEGG (Kyoto Encyclopedia of Genes and Genomes).

### Example 2

Broiler Trials Comparing the Effect Between a Diet with Added Ronozyme ProAct Protease and a Control Diet

The trial was based on completely randomized block design. Male Hubbard x Ross chickens were used and the trial lasted 42 days. The starter, grower and finisher diets contained respectively 583, 654 and 694 g/kg of corn, 300, 204, 85 169 g/kg of soybean meal and 0, 100 and 100 g/kg of corn DDGS. In the treated diets, protease commercially available under the name Ronozyme ProAct at DSM Nutritional Products was dosed at 200 g/tonne of feed.

Samples of gastrointestinal (GIT) content was collected from the cecum at day 21 of an animal trial. Each GIT sample was extracted for DNA using MoBio Powersoil kit. DNA was then sequenced on an Illumina HiSeq 3000 to produce >2 million random reads of 100 bp representing the DNA of microbes in the GIT. Reads from the sequencing run were aligned using the Burrows-Wheeler alignment algorithm against a reference database of genes that had been previously annotated using KEGG.

The study of Example 2 shows that chickens which were fed with Ronozyme ProAct have a down-regulated LEE and non-LEE pathway.

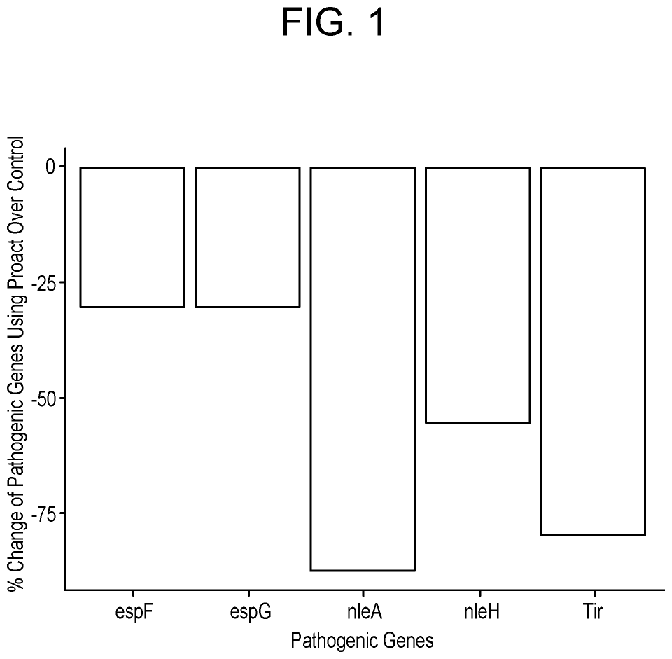

FIG. 1 represents the percent change in LEE genes of espG, espF, and Tir, and non-LEE genes of nleA and nleH in the microbiome of broilers which were fed ProAct™ against those broilers which were fed the same diet except for ProAct™. It shows that Proact™ has helped to reduce the relative presence of pathogenic genes in the GIT of broilers. The reduction of these genes in the ProAct treated group is over 25% for both LEE genes and non-LEE genes. The highest reduction, such as nleA gene, is more than 75%.

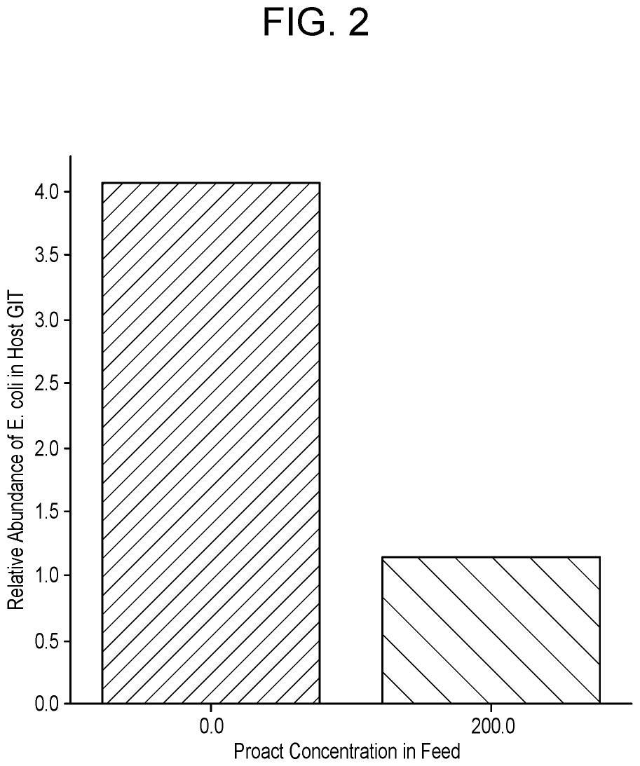

In the same study, changes in the relative abundance of E. coli were measured by aligning reads as described above against taxonomic database included with MetaPhlan2. For this, MetaPhlan2 was used to describe the taxonomic content of the microbiome derived from the GIT. MetaPhlan2 uses Glade-specific taxonomic markers to identify the relative abundance of different organisms within a sample. FIG. 2 below shows that in broilers which were fed a control diet, there is a greater relative abundance of E. coli in the makeup of the microbial composition than in broilers which were fed ProAct™ diet. This shows that protease ProAct™ helps to reduce the relative abundance of E. coli in the GIT of broilers. In the control group, the % of population of E. coli in the microbiome is at about 4%. In the ProAct™ treated group, the % of population of E. coli was reduced to about 1.25%. The reduction of E. coli is thus more than 2 folds.

### Example 3

Swine Trial Comparing the Effect Between a Diet with Added Superoxide Dismutase and Control Diet

The trial used domestic swine and it lasted 42 days. The diet comprises cereal grains as basic ingredients In the treated diets, superoxide dismutase, which is made by DSM Nutritional Products, was dosed at 2000 IU of feed.

Samples of gastrointestinal (GIT) content was collected from the cecum at days 0, 14 and 21 of an animal trial. Each GIT sample was extracted for DNA using MoBio Powersoil kit. DNA was then sequenced on an Illumina HiSeq 3000 to produce >2 million random reads of 100 bp representing the DNA of microbes in the GIT. Reads from the sequencing run were aligned using the Burrows-Wheeler alignment algorithm against a reference database of genes that had been previously annotated using KEGG.

The study of Example 3 shows that chickens which were fed with feed enzyme additive such as superoxide dismutase has a down-regulated LEE and non-LEE pathway.

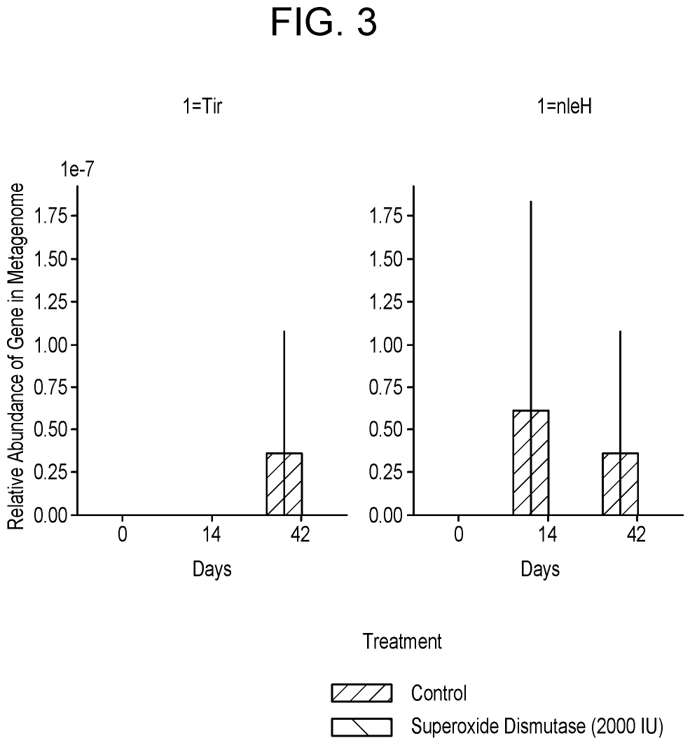

FIG. 3 shows the relative abundance of LEE gene Tir and non-LEE gene nleH in the microbiome of animals which was fed superoxide dismutase against that os those animals fed the same diet but without the addition of superoxide dismutase. It shows that at days 14 and 42, the relative abundance of LEE gene Tir and non-LEE gene nleH comparing to day 0 has increased in the control group, but such abundance has remained unchanged in the superoxide dismutase treated group. It shows that superoxide dismutase helped to reduce the abundance of LEE genes and non-LEE genes in the microbiome of animals.

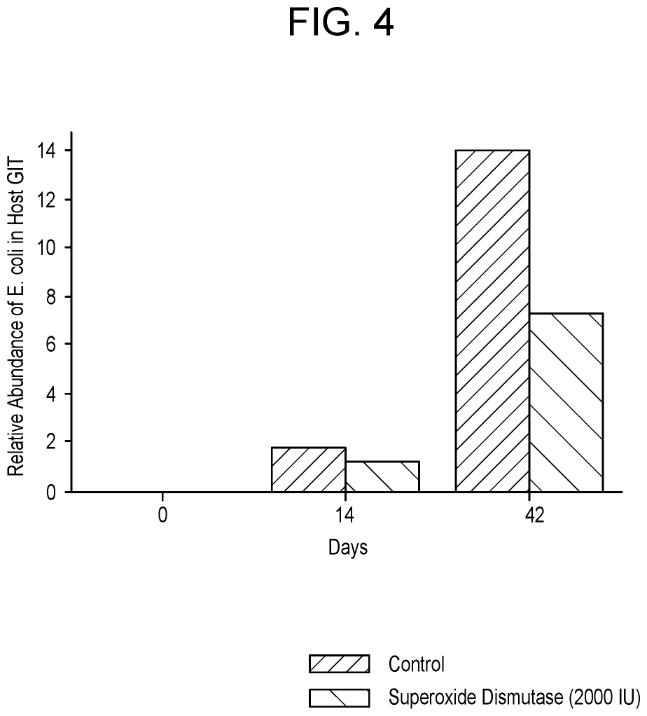

In the same study, changes in the relative abundance of E. coli were measured by aligning reads as described above against taxonomic database KEGG Orthology. For this, MetaPhlan2 was used to describe the taxonomic content of the microbiome derived from the GIT. FIG. 4 shows that in swine fed a control diet, there is a greater relative abundance of E. coli in the makeup of the microbial composition than in swine fed superoxide dismutase. This indicates that the addition of superoxide dismutase can help reduce the relative abundance of E. coli.

### Example 4

Turkey Trial Comparing the Effect Between a Diet with Added Lysozyme and Control Diet

In this experiment, domestic turkey were fed with commercial turkey feed with and without added Lysozyme Balancius™. The trial lasted 63 days. Samples of gastrointestinal (GIT) content was collected from the ileum and cecum at days 42 and 63 of the trial. Each GIT sample was extracted for DNA using MoBio Powersoil kit. DNA was then sequenced on an Illumina HiSeq 3000 to produce >2 million random reads of 100 bp representing the DNA of microbes in the GIT. Reads from the sequencing run were aligned using the Burrows-Wheeler alignment algorithm against a reference database of genes that had been previously annotated using KEGG (Kyoto Encyclopedia of Genes and Genomes).

The study of Example 4 shows that turkeys which were fed with Lysozyme Balancius™ has a down-regulated LEE and non-LEE pathway towards the end of their growing period.

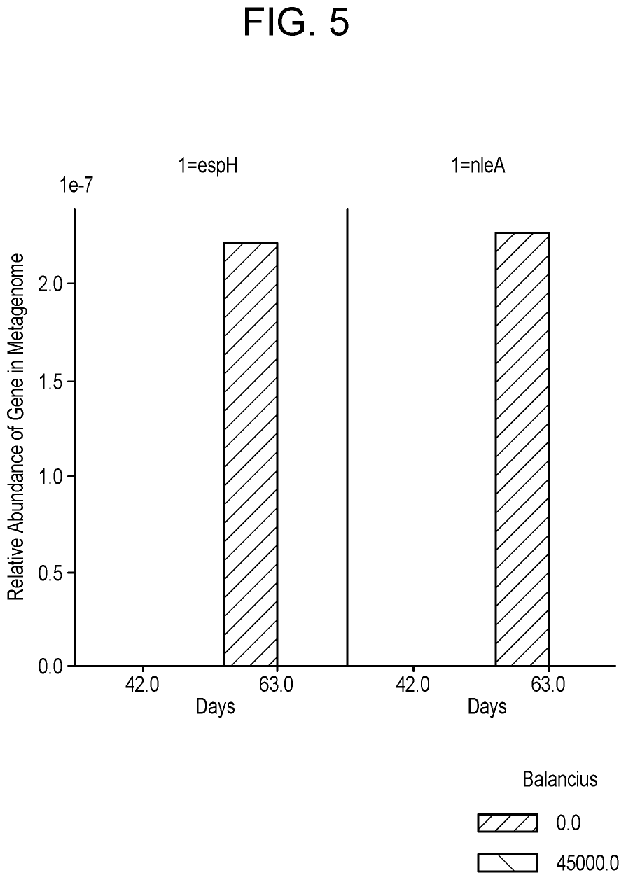

Lysozyme Balancius™ was designed to break down peptidoglycans (PGNs) in bacterial cell debris, thus releasing nutrients and unlocking a hidden potential in gastrointestinal functionality. FIG. 5 shows the relative abundance of LEE and non-LEE genes in the metagenome across the various days and broken out by turkeys on a diet that contains lysozyme Balancius™ at 45,000 IU while the control case did not. It is shown that the continued use of Balancius™ can reduce detectable LEE and non-LEE genes close to the end of the trial, whereas control turkeys showed a marked increase across several LEE and non-LEE genes, suggesting the control group turkeys are more susceptible to pathogenic infection at the end of the trial.

### Example 5

In this experiment, domestic laying hens are fed with commercial poultry feed with and without added Ronozyme ProAct protease. The trial lasted 42 days. In the treated diets, protease commercially available under the name Ronozyme ProAct at DSM Nutritional Products was dosed at 200 g/tonne of feed.

Samples of gastrointestinal (GIT) content was collected from the cecum at day 21 of an animal trial. Each GIT sample was extracted for DNA using MoBio Powersoil kit. DNA was then sequenced on an Illumina HiSeq 3000 to produce >2 million random reads of 100 bp representing the DNA of microbes in the GIT. Using a taxonomic identification program like MetaPhlanv3, microbiota is identified in the samples, whereas an alignment algorithm like the Burrows-Wheeler alignment algorithm is used against a database of genes. Copy number of Bacteroides thetaiotaomicron market gene and E. coli LEE and non-LEE genes are measured, in both the enzyme diet treated group and the control group.

The study of Example 5 shows that hens which are fed with Ronozyme ProAct has reduced copy number of Bacteroides thetaiotaomicron market gene in the enzyme diet treated group when comparing to the control group. It is also observed that the LEE and non-LEE pathway of E. coli is down regulated in the enzyme diet treated group when comparing to the control group.

1. A method for reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, comprising feeding said animal with one of more of the following feed additives: N-acetyl-muramidase, protease, superoxide dismutase and/or catalase, wherein the population of exogenous LEE genes and non-LEE pathogenic genes is reduced by at least 10% lower than that of a control animal which is fed with the same diet except for said feed additives;
preferably wherein the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide;
preferably wherein the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and
preferably wherein the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f′) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (1″) a fragment of the polypeptide of (a″), (b″), (c″), (d″), (e″), (f′), (g″), (h″), (i″), (j″), or (k″) that has superoxide dismutase activity.

2. The method of claim 1, wherein said population of exogenous LEE genes and non-LEE pathogenic genes is measured as % ratio of the combined copy numbers of LEE genes and non-LEE genes detected within the microbiome of said animal vs. the total copy number of genes detected within said microbiome.

3. The method of claim 2, wherein said microbiome is collected from either a fecal sample of the animal or a sample collected within the GIT of the animal.

4. The method of claim 3, wherein said gene copy number measurement is performed by RT-PCR counting, full length 16S RNA sequencing, or Metagenomic DNA sequencing.

5. The method of claim 1, wherein said animal is a production animal.

6. The method of claim 1, wherein said LEE genes comprise: Tir, Map, EspB, EspF, EspG, EspH, and EspZ.

7. The method of claim 1, wherein said non-LEE pathogenic genes comprise: EspG2, EspJ, EspM1/2, EspT, EspW, Cif, NleA, NleB, NleC, NleD, NleE, NleF, and NleH.

8. The method of claim 1, wherein said feed additive comprises further feed enzymes and/or further gut health enzymes.

9. The method of claim 8, wherein said further feed enzymes comprise protease, phytases, xylanases, cellulases, mannanases, α-galactosidases, pectinases, and amylases.

10. The method of claim 9, wherein said further gut health enzymes comprise N-acetyl-muramidase, superoxide dismutase (SOD), and/or catalase.

11. The method of claim 8, wherein the concentration of said enzymes is between 50 and 1000 g/tonne of the feed to be given to the group of production animals.

12. The method of claim 1, wherein said production animals are: broiler chickens, turkeys, ducks, layers, piglets, grower pigs, finisher pigs, and sows.

13. A method for reducing the population of Bacteroides thetaiotaomicron in the gastrointestinal tract (GIT) of an animal, comprising feeding said animal with one of more of the following feed additives: N-acetyl-muramidase, protease, superoxide dismutase and/or catalase, wherein the population of Bacteroides thetaiotaomicron is reduced by at least 10% lower than that of a control animal which is fed with the same diet except for said feed additives;
preferably wherein the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide;
preferably wherein the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and
preferably wherein the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f′) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (1″) a fragment of the polypeptide of (a″), (b″), (c″), (d″), (e″), (f′), (g″), (h″), (i″), (j″), or (k″) that has superoxide dismutase activity.

14. The method of claim 13, wherein said population of Bacteroides thetaiotaomicron is measured as % ratio of the population of Bacteroides thetaiotaomicron detected within the microbiome of said animal against the total population of microbes within said microbiome.

15. The method of claim 14, wherein said microbiome is collected from the fecal sample of the animal or a sample collected within the GIT of the animal.

16. The method of claim 15, wherein population measurement is performed by RT-PCT counting, full length 16S RNA sequencing, or Metagenomic DNA sequencing.

17. The method of claim 13, wherein said animal is a production animal.

18. A method of reducing the population of E. coli in the gastrointestinal tract (GIT) of an animal, comprising feeding said animal with one of more of the following feed additives: N-acetyl-muramidase, protease, superoxide dismutase and/or catalase, wherein the systemic inflammation and/or local inflammation of the animal is reduced by at least 10% lower than that of a control animal which are fed with the same diet except for said feed additives;
preferably wherein the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide;
preferably wherein the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and
preferably wherein the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f′) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (1″) a fragment of the polypeptide of (a″), (b″), (c″), (d″), (e″), (f′), (g″), (h″), (i″), (j″), or (k″) that has superoxide dismutase activity.

19. The method of claim 18, wherein said E. coli is pathogenic E. coli.

20. The method of claim 19, wherein the pathogenic E. coli comprises EPEC, EHEC, and APEC.

21. The method of claim 20, wherein said population of E. coli in the GIT of the animal is measured as % of copy number of E. coli marker genes within the microbiome of said animal against the total copy number of bacterial marker genes detected within said microbiome.

22. The method of claim 21, wherein said microbiome is collected from the fecal sample of the animal or a sample collected within the GIT of the animal.

23. The method of claim 22, wherein said measurement is performed by RT-PCT counting, full length 16S RNA sequencing, or Metagenomic DNA sequencing.

24. The method of claim 18, wherein said animal is a production animal.

25. A method for reducing systemic inflammation and/or local inflammation of an animal caused by E. coli infection, comprising feeding said animal with one of more of the following feed additives: N-acetyl-muramidase, protease, superoxide dismutase and/or catalase, wherein the systemic inflammation and/or local inflammation of the animal is reduced by at least 10% lower than that of a control animal which are fed with the same diet except for said feed additives;
preferably wherein the N-acetyl-muramidase is selected from the group consisting of: (a) a polypeptide having at least 80% sequence identity to any one of SEQ ID NOs: 1-71; (b) a variant of a polypeptide having any one of SEQ ID NOs: 1-71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions; (c) a polypeptide comprising the polypeptide of (a) or (b) and a N-terminal and/or C-terminal extension of between 1 and 10 amino acids; and (d) a fragment of a polypeptide of (a) or (b) having muramidase activity and having at least 90% of the length of the mature polypeptide;
preferably wherein the protease is selected from the group consisting of: (a′) a polypeptide having a sequence identity of at least 70% to any one of SEQ ID NOs 72-76; (b′) a variant of any one of SEQ ID NOs: 72-76, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; (c′) a polypeptide comprising the polypeptide of (a′) or (b′) and a N-terminal and/or C-terminal His-tag and/or HQ-tag; (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and (e′) a fragment of the polypeptide of (a′) or (b′) having protease activity and having at least 90% of the length of the mature polypeptide; and
preferably wherein the superoxide dismutase is selected from the group consisting of: (a″) a polypeptide having at least 75% sequence identity to SEQ ID NO: 77; (b″) a polypeptide having at least 75% sequence identity to SEQ ID NO: 78; (c″) a polypeptide having at least 75% sequence identity to SEQ ID NO: 79; (d″) a polypeptide having at least 75% sequence identity to SEQ ID NO: 80; (e″) a polypeptide having at least 85% sequence identity to amino acid residues 52 to 170 of SEQ ID NO: 77; (f′) a polypeptide having at least 85% sequence identity to amino acid residues 55 to 136 of SEQ ID NO: 78; (g″) a polypeptide having at least 85% sequence identity to amino acid residues 12 to 149 of SEQ ID NO: 79; (h″) a polypeptide having at least 85% sequence identity to amino acid residues 47 to 179 of SEQ ID NO: 80; (i″) a polypeptide encoded by a polynucleotide having at least 80% sequence identity to the sequence of SEQ ID NO: 81; (j″) a polypeptide encoded by a polynucleotide that hybridizes under medium-high stringency conditions with coding sequence of SEQ ID NO: 81 or the full-length complement thereof; (k″) a variant of the polypeptide of SEQ ID NO: 77, or SEQ ID NO: 78, or SEQ ID NO: 79, or SEQ ID NO: 80, or amino acid residues 52 to 170 of SEQ ID NO: 77, or amino acid residues 55 to 136 of SEQ ID NO: 78, or amino acid residues 12 to 149 of SEQ ID NO: 79, or amino acid residues 47 to 179 of SEQ ID NO: 80 comprising a substitution, deletion, and/or insertion at one or more positions and that has superoxide dismutase activity; and (1″) a fragment of the polypeptide of (a″), (b″), (c″), (d″), (e″), (f′), (g″), (h″), (i″), (j″), or (k″) that has superoxide dismutase activity.

26. The method of claim 25, wherein said reduction of inflammation is measured as % ratio of the copy number of LEE and non-LEE genes detected within the microbiome of said animal against the total copy number of genes detected within said microbiome.

27. The method of claim 26, wherein said microbiome is collected from the fecal sample of the animal or a sample collected within the GIT of the animal.

28. The method of claim 27, wherein said measurement is performed by RT-PCT counting, full length 16S RNA sequencing, or Metagenomic DNA sequencing.

29. The method of claim 25, wherein said animal is a production animal.

30. Use of feed enzymes, in particular N-acetyl-muramidase, protease, superoxide dismutase and/or catalase, for
a) reducing the population of exogenous locus for enterocyte effacement (LEE) genes and exogenous non-LEE pathogenic genes of Enterohemorrhagic E. coli (EHEC), Enteropathogenic E. coli (EPEC), and Avian Pathogenic E. coli (APEC) in the gastrointestinal tract (GIT) of an animal, wherein the population of exogenous LEE genes and non-LEE pathogenic genes is reduced by at least 10% lower than that of a control animal which is fed with the same diet except for said feed additives;
b) reducing the population of Bacteroides thetaiotaomicron in the gastrointestinal tract (GIT) of an animal, wherein the population of Bacteroides thetaiotaomicron is reduced by at least 10% lower than that of a control animal which is fed with the same diet except for said feed additives;
c) reducing the population of E. coli in the gastrointestinal tract (GIT) of an animal, wherein the systemic inflammation and/or local inflammation of the animal is reduced by at least 10% lower than that of a control animal which are fed with the same diet except for said feed additives; and/or
d) reducing systemic inflammation and/or local inflammation of an animal caused by E. coli infection, wherein the systemic inflammation and/or local inflammation of the animal is reduced by at least 10% lower than that of a control animal which are fed with the same diet except for said feed additives.
